(19) United States
(12) Patent Application Publication
(10) Pub. No.: US 2018/0094320 A1
(43) Pub. Date: Apr. 5, 2018

(54) IN VITRO DRUG METABOLISM REAGENT AND USES THEREOF

(71) Applicant: Albert LI, (US)

(72) Inventor: Albert LI, Columbia, MD (US)

(73) Assignee: Albert LI, Columbia, MD (US)

(21) Appl. No.: 15/723,463

(22) Filed: Oct. 3, 2017

Related U.S. Application Data
(60) Provisional application No. 62/403,435, filed on Oct. 3, 2016.

(51) Int. Cl. C12Q 1/68; G01N 33/50

(52) U.S. Cl. CPC C12Q 1/6883; G01N 33/5038; G01N 33/5067; G01N 33/5014

(57) ABSTRACT

The present disclosure provides an in vitro reagent for evaluating xenobiotic metabolism in a cell culture based assay. The in vitro reagent is an admixture of metabolically competent cells and exogenous drug metabolizing enzyme co-factors follow by cryopreservation in the absence of cryopreservation agent so that the cells would be rendered permeable upon thawing due to plasma membrane disruption (while maintaining the integrity of organelles). The permeabilized plasma membranes allow ready diffusion of the exogenous cofactors into the cells to enhance the activities of cellular drug metabolizing enzymes. Addition of a xenobiotic test compound to the thawed in vitro reagent allows metabolism of the test compound by the metabolically competent cells, with metabolites readily diffusible outside the cells due to the permeabilized plasma membranes.

### CROSS-REFERENCE TO RELATED APPLICATIONS

This application claims the benefit of U.S. Provisional Patent Application No. 62/403,435; filed on 3 Oct. 2016, the content of which is incorporated herein by reference in its entirety.

### FIELD OF THE INVENTION

The disclosed inventions relate generally to cryopreserved mixture of metabolically competent cells with a permeabilized plasma membrane and supplemented metabolic enzyme cofactors for use in the evaluation of xenobiotic metabolism.

### BACKGROUND OF THE INVENTION

The invention relates to in vitro reagents for evaluating xenobiotic metabolism, methods of manufacturing the reagent and their methods of use for evaluating xenobiotic metabolism and as an exogenous metabolic system for cell culture assays with metabolic incompetent cells (cells that are not capable of xenobiotic metabolism).

Drug metabolism is an important aspect of drug development. As a drug is administered to the human body, the parent drug is subjected to metabolism by the small intestine before absorption into the portal circulation and subjected to metabolism by the liver. Drug metabolism is critical to the system half-live of the parent drug due to metabolic elimination, toxicity due to the toxicity of the parent drug and metabolites, and efficacy due to the pharmacological effects of the parent drug and metabolites. Due to species difference in drug metabolism, studies with nonhuman animals do not always provide information useful for the assessment of human outcomes. This is overcome by the use of in vitro human-based experimental systems. Evaluation of drug metabolism using in vitro human-based system is now routinely practiced in drug development.

There are myriad assays using bacterial and mammalian cell cultures to estimate biological effects of a substance in the human body. A major drawback of these assays is that the target cells do not possess xenobiotic drug metabolizing enzymes, therefore can only detect results of the parent test substance, but not the metabolites as would occur in the human body. To overcome this major defect of in vitro experimental systems, an exogenous metabolic system is needed to provide xenobiotic metabolism to these cell culture assays. Exogenous metabolic systems are presently not readily available except for the use of rat liver homogenate in genotoxicity assays which may yield results not relevant to human due to species difference.

In vitro drug metabolism systems are mainly derived from the human liver as it is a major organ for drug metabolism. Commonly used in vitro experimental systems are liver homogenates and intact hepatocytes. For liver homogenate systems, the liver is disrupted by homogenization. In general, the homogenate is first centrifuged at a relative low speed of 9000 or 1000×g, the resulting supernatant contains both cytosolic and microsomal enzyme and can be used to evaluate both phase I oxidation and phase II conjugation. The supernatant can be centrifuged at a high speed (100,000×g), resulting in a pellet of endoplasmic reticulum, also referred to as microsomes, which contain one of the most important drug metabolizing enzyme, cytochrome P450. Liver homogenate supernatant, commonly referred to as S-9 or S-10 (supernatant form after centrifugation at 9,000×g or 10,000×g, respectively) and the microsomes are the most commonly used in vitro cell-free experimental system for the evaluation of drug metabolism. The liver homogenate systems represent a convenient tool for the evaluation of drug metabolism. Use of the liver homogenate systems such as S9 or microsomes requires the additional of exogenous cofactors to be added at the time of experimentation. A major disadvantage of the homogenate system is that tissue disruption during the preparation of the homogenates may lead to non-physiological findings that may not represent events in human in vivo.

Another commonly used in vitro hepatic drug metabolic system is intact hepatocytes. Hepatocytes are isolated from a liver by enzyme digestion (e.g. collagenase digestion) and purified by centrifugation, usually a relative low speed (e.g 50×g) to remove non-hepatocytes (e.g. kupffer cells and endothelial cells). The 50×g pellet contains mainly the parenchymal cells or hepatocytes which are the cells responsible for drug metabolism in the liver. Hepatocytes can be used directly after isolation or cryopreserved and stored in liquid nitrogen for use. Successful cryopreservation of hepatocytes to retain viability and function is one of the major reasons for routine application of hepatocytes in experimentation. Cryopreserved hepatocytes from human and non-human animals are commercially available. A scientist can use hepatocytes for experimentation by purchasing the cryopreserved hepatocytes and store them in liquid nitrogen. On the day of experimentation, the cryopreserved hepatocytes are thawed and quantified for viability and cell number, followed by use in experimentation. Cryopreservation of hepatocytes allow this experimental system to be used routinely for the evaluation of drug metabolism, eliminating the need to prepare hepatocytes for each study.

A major difference between the use of liver homogenate-based systems (S9, S10, microsomes) and hepatocytes is that liver homogenates need to be supplemented with enzyme cofactors such as NADPH for oxidative metabolism (e.g. hydroxylation of midazolam by P450 isoform 3A4 to 1-hydroxymidazolam), UDPGA for glucuronidation (e.g. formation of 7-OH coumarin glucuronide from 7-OH-coumarin), and PAPS for sulfation (e.g. formation of 7-OH-coumarin sulfate from 7-OH-coumarin). S9 or S10 can be used to evaluate both phase 1 oxidation and conjugation using all three cofactors; microsomes are used mainly for oxidation as most phase II conjugating enzymes are in the cytosol and not in the microsomal membranes. Viable, intact hepatocytes contain endogenous cofactors, and a full complement of hepatic drug metabolizing enzymes. Hepatocytes therefore are considered the gold standard which can be used directly, without adding cofactors, for all hepatic metabolism pathways.

This invention is intended to overcome technical challenges required for the application of hepatocytes in drug metabolism. The use of hepatocytes requires specific laboratory equipment such as liquid nitrogen storage system, as the cells need to be store at <−150 deg. C. for long term stability, unlike liver homogenate systems that can be stored in a commonly used laboratory freezer at −20 or −80 deg. C. After thawing, the hepatocytes require to be purified (e.g. using the Cryopreserved Hepatocyte Recovery Medium, GIBCO or Universal Cryopreservation Recovery Medium, In Vitro ADMET Laboratories), reconstituted, quantified for cell viability, and adjustment of cell density before addition to the reaction mixture for the evaluation of drug metabolism. Researchers need to be trained with specific skills to work with hepatocytes as the cells are fragile and can be easily damaged by inexperience practitioners. Lastly, hepatocyte isolation and cryopreservation may lead to decreases in cofactors required for drug metabolism, thereby leading to activity lower than that in vivo.

A second organ important for drug metabolism are intestinal mucosal cells or enterocytes. Enterocytes are now known to contain key drug metabolizing enzymes. Enterocytes can be routinely isolated and used for enteric drug metabolism studies as intact cells, akin to intact hepatocytes for the evaluation of hepatic drug metabolism. Enterocyte S9 and enterocyte microsomes are also used as in vitro systems for the evaluation of enteric drug metabolism, similar to hepatic S9 and microsomes for hepatic metabolism. Enterocyte homogenates and intact enterocytes have similar advantages and drawbacks as described above for the corresponding hepatic systems.

The invention disclosed herein addresses those deficiencies and provides an improvement for evaluating xenobiotic metabolism using the present in vitro reagent; the invention possesses the advantages of both the tissue homogenates and intact cell models for the evaluation of drug metabolism. For metabolically competent cells isolated from human or animal organs, this invention produces a reagent that eliminates the need of a liquid nitrogen freezer. The reagent can be used immediately after thawing, eliminating the multiple cell manipulation steps needed for fragile cells such as cryopreserved hepatocytes. Supplementation of cells such as hepatocytes with cofactors (which is not a useful approach as the exogenous cofactors have limited permeability to plasma membranes and therefore not assessable to the cellular drug metabolizing enzymes) in the reagent allows restoration of the cofactors that have been diminished due to the isolation and cryopreservation processes to physiologically relevant in vivo levels.

### SUMMARY OF THE INVENTION

Herein are provided an in vitro reagent, manufacturing of the in vitro reagent and methods for evaluating xenobiotic metabolism using the in vitro reagent. In embodiments provided herein is an in vitro reagent wherein the reagent is a cryopreserved mixture comprising metabolically competent cells wherein cell membranes are permeabilized upon thawing, exogenous drug metabolizing enzyme co-factors, and a buffered solution (e.g., cell culture medium), wherein the reagent does not comprise a cryopreservative agent. In embodiments, the in vitro reagent is thawed wherein the mixture comprises metabolically competent cells with permeabilized plasma membranes. In embodiments, the in vitro reagent is further added to and comprises a metabolically incompetent mammalian or microbial culture, wherein the reagent provides exogenous metabolic functions for evaluation of a physiological effect of metabolites of a xenobiotic.

In embodiments, the in vitro reagent comprises co-factors required for biochemical pathways and xenobiotic metabolism. In certain embodiments, the drug metabolizing enzyme co-factors are selected from β-nicotinamide adenine dinucleotide 2′-phosphate (NADPH; cofactor for oxidative metabolism), uridine 5′-diphosphoglucuronic acid (UDPGA; cofactor for glucuronidation), 3′-phosphoadenosine 5′-phosphosulfate (PAPS; cofactor for sulfation), N-acetyl coenzyme A (cofactor for N-acetylation), L-glutathione (cofactor for glutathione conjugation), s-adenosyl methionine, amino acids, and carnitine.

In embodiments, the metabolically competent cells are primary cells or cell lines, and may be human, rat, monkey, dog, mammals, avian or non-mammalian. In certain embodiments, the metabolically competent cells are from organs responsible for drug metabolism in vivo, such as liver (hepatocytes), intestine (enterocytes) and kidney (renal cells). In embodiments, the metabolically competent cells (primary cells) are pooled from more than one donor. The metabolically competent cells can also be cell lines engineered to contain cytochrome P450 isoforms. In embodiments, the metabolically competent cells comprise drug metabolizing enzyme (DME) activities.

In embodiments, the reagent is provided in a kit and further comprises instructions for evaluating xenobiotic metabolism using the reagent.

In certain embodiments, provided herein is an in vitro reagent for evaluating xenobiotic metabolism, wherein the reagent is a cryopreserved mixture comprising: a) metabolically competent cells selected from hepatocytes or enterocytes wherein cell membranes of the hepatocytes or enterocytes are permeabilized upon thawing; b) exogenous drug metabolizing enzyme co-factors selected from β-Nicotinamide adenine dinucleotide 2′-phosphate (NADPH), Uridine 5′-diphosphoglucuronic acid (UDPGA), 3′-Phosphoadenosine 5′-phosphosulfate (PAPS), N-acetyl coenzyme A, s-adenosyl methionine, amino acids, carnitine, and L-glutathione; and, c) a buffered solution, wherein the reagent does not comprise a cryopreservative agent.

In certain embodiments, provided herein are methods for manufacturing the in vitro reagent for evaluating xenobiotic metabolism. In embodiments, the method comprises permeabilizing metabolically competent cells, adding exogenous drug metabolizing enzyme co-factors either before or after the metabolically competent cells are permeabilized and providing a buffered solution (e.g., cell culture medium), wherein the reagent does not comprise a cryopreservative agent. In embodiments, the method further comprises storing the reagent frozen at a temperature of −10° C., or lower, such as at a temperature from about −10° C. to about −80° C.

In embodiments, the metabolically competent cells are permeabilized by freezing in the absence of a cryopreservative agent leading to permeabilization upon thawing. In embodiments, the reagent is not stored frozen in liquid nitrogen. In certain embodiments, the permeabilized metabolically competent cells are not centrifuged prior to use. In other embodiments, the permeabilized metabolically competent cells are not counted prior to use.

Provided herein are methods for manufacturing the present in vitro reagent, comprising: a) combining intact metabolically competent cells with exogenous drug metabolizing enzyme co-factors in a cell culture medium to form a cell mixture; b) freezing the cell mixture at a temperature from about −10° C. to about −80° C., wherein the cell mixture does not comprise a cryopreservative agent; and, c) thawing the cell mixture to form the in vitro reagent wherein cell membranes of the metabolically competent cells are permeabilized via thawing.

In embodiments, the in vitro reagent is used to evaluate xenobiotic metabolism after thawing without the step of centrifuging or cell counting.

In certain embodiments, provided herein are methods of determining metabolism of a test compound, wherein the method comprises providing a present cyropreserved in vitro reagent, thawing the present in vitro reagent and placing in a cell culture vessel; introducing the xenobiotic test compound into the cell culture vessel, incubating the test compound for 0.5 h to 10 days at 33-40° C. and performing an end point assay of the in vitro reagent or cell culture medium to determine metabolism of the xenobiotic test compound. In embodiments, the cell culture vessel comprising the in vitro reagent and test compound is incubated at 37° C.

In embodiments, the end point assay comprises an Ames Salmonella histidine reversion assay (Ames test) for genotoxicity, a mammalian or non-mammalian genotoxicity assay, a mammalian or non-mammalian cytotoxicity assay, and/or a mammalian or non-mammalian pharmacological assay.

In embodiments, the xenobiotic agent is a drug or drug candidate, an industrial chemical and/or environmental pollutant. In certain embodiments, the drug or drug candidate is selected from the group consisting of an organic compound, an inorganic compound, a hormone, a growth factor, a cytokine, a reception, an antibody, an enzyme, a peptide, an aptomer and a vaccine.

In embodiments, step b) (thawing the in vitro reagent and placing in a cell culture vessel) of the present methods for determining metabolism of a test compound does not further comprise a centrifugation or cell counting step. In certain embodiments, the present method does not comprise use of a microscope before step e) (performing an end point assay of the in vitro reagent or cell culture medium to determine metabolism of the xenobiotic test compound).

In embodiments, the cryopreserved in vitro reagent of step a) comprises exogenous drug metabolizing enzyme co-factors used to select metabolism pathways. See Example 13. In certain embodiments, the present method for determining metabolism of a test compound comprises adding a drug or drug candidate at cytotoxic concentrations for identifying and profiling metabolites. See Example 12. In certain embodiments, the present method for determining metabolism of a test compound comprises evaluation of complete phase 1 oxidation and phase 2 conjugation of metabolites with the present reagent of step a) (providing the present cryopreserved in vitro reagent). See Example 8. In certain embodiments, the present methods for determining metabolism of a test compound further comprises evaluating metabolic stability, metabolite profiling and identification, enzyme inhibition or metabolic activation of prototoxicants or pro-mutagens.

In embodiments, the time to perform combined steps a) (providing a present cryopreserved in vitro reagent), b) (thawing the in vitro reagent and placing in a cell culture vessel) and c) (introducing the xenobiotic test compound into the cell culture vessel) takes about 10 minutes or less, such as from about 2 minutes to about 6 minutes.

In embodiments provided herein is an exogenous metabolic system for evaluation of a physiological effect of metabolites of a xenobiotic test compound, wherein the system comprises a cell culture medium admixture of metabolically competent cells comprising permeabilized plasma membranes and exogenous drug metabolizing enzyme co-factors; and metabolically incompetent target cells. The target cells are used to evaluate the metabolites produced by the metabolically competent cells, such as those salmonella bacterial cells used in an Ames test; reporter cell lines for the evaluation of cytotoxicity or pharmacologic effects.

In embodiments, the system further comprises a cell culture vessel comprising one or more assay wells, wherein the assay wells comprise the cell culture medium admixture. In embodiments, each assay well is divided into a first and second chamber, wherein a first chamber comprises the cell culture medium admixture and the second chamber comprises the metabolically incompetent target cells provided the metabolically incompetent target cells are in fluid communication with the cell culture medium admixture. In embodiments, the first and second chamber are separated by a porous membrane that is liquid-permeable and cell-impermeable.

In embodiments, the metabolically incompetent target cells may be mammalian or non-mammalian, such as bacteria.

### BRIEF DESCRIPTION OF THE DRAWINGS

FIG. 1 provides results from metabolism of 7-ethoxycoumarin by human enterocytes with or without exogenous drug metabolizing enzyme co-factors and with or without permeabilization. Highest activity was observed with permeabilized enterocytes with cofactors (Group D; present in vitro reagent). See Example 4

FIG. 2A and FIG. 2B provide measurement of the major P450 isoform activities as a function of cell concentrations. The linear concentration versus activity curves demonstrate that permeabilized hepatocytes can be used routinely as an in vitro drug metabolism system for scientific investigations.

FIGS. 3A and 3B show P450 isoform-selective substrate metabolism in intact human hepatocytes and permeabilized human hepatocytes over a time period of 30 minutes to 240 minutes.

FIG. 4 shows non-P450 pathway-selective substrate metabolism in intact human hepatocytes and permeabilized human hepatocytes over a time period of 30 minutes to 240 minutes.

FIG. 5 shows phase 1 oxidation and phase 2 conjugation metabolism of coumarin.

FIG. 6 shows coumarin metabolite formation and ethoxycoumarin formation in intact human hepatocytes and permeabilized human hepatocytes demonstrating sequential drug metabolism in the permeabilized human hepatocytes measured at 30 min., 60 min., 120 min. and 240 min.

FIG. 7 shows a comparison of 16 drug metabolizing enzyme (DME)-selective substrates and measurement of metabolites in intact human hepatocytes (open bars) and permeabilized human hepatocytes (solid bars). The permeabilized human hepatocytes showed similar or higher activity than the intact human hepatocytes in the metabolism of 16 pathway-selective DME pathways.

FIG. 8 shows the advantages of permeabilized hepatocytes and permeabilized enterocytes as compared to cell free systems (S9 and microsomes) or intact hepatocytes and intact enterocytes. The permeabilized hepatocytes and permeabilized enterocytes provide complete representation of organelles as intact hepatocytes and enterocytes.

FIG. 9 shows the ease of use of the permeabilized hepatocytes and permeabilized enterocytes as compared to cell free systems and intact hepatocytes and intact enterocytes.

FIG. 10 shows metabolic stability screening wherein the present permeabilized human hepatocytes (filled symbols; y=0.79x; R²=0.4067) accurately predict human hepatic clearance in vivo of drugs with known in vivo hepatic clearance as compared to intact hepatocytes (open symbols; y=0.7375x; R²=0.13). See Table 4 for a list of the drugs tested and observed results.

FIG. 11 shows Acetaminophen metabolism (glucuronidation, sulfation and GSH conjugation to the oxidative metabolite NAPQI) at nontoxic (10 mM) and cytotoxic (100 and 200 mM) concentrations in both intact human hepatocytes (open bars) and permeabilized human hepatocytes (solid bars). The permeabilized human hepatocytes can be incubated with high, cytotoxic concentrations of a xenobiotic (e.g. drug or drug candidate) for the generation of metabolites for identification and profiling while significant reduction of metabolite formation was observed in intact hepatocytes due to cytotoxicity.

FIG. 12 shows cofactor-directed pathway selection scheme of coumarin metabolism.

FIG. 13 shows cofactor-directed pathway (no cofactors; NADPH; NADP+PAPS; and, NADPH+UGPGA) selection of coumarin metabolism (7-HC; 7-HCS; and, 7-HCG) in permeabilized human hepatocytes. Selection of exogenous drug metabolizing enzyme co-factors can be used to direct metabolism to specific pathways in the present permeabilized human hepatocytes.

FIG. 14 shows a scheme of the prototoxicant Acetaminophen activation.

FIG. 15 shows a scheme of the prototoxicant Cyclophosphamide activation.

FIG. 16 shows the activation of Acetaminophen using permeabilized human hepatocytes with boiled hepatocytes and no hepatocytes as controls.

FIG. 17 shows the activation of Cyclophosphamide and Ifosfamine using permeabilized human hepatocytes with boiled hepatocytes and no hepatocytes as controls.

FIG. 18 shows the versatility of using permeabilized hepatocytes and permeabilized enterocytes as compared to cell free systems (S9 and microsomes) or intact hepatocytes and intact enterocytes.

FIG. 19 shows the steps of using permeabilized hepatocytes and permeabilized enterocytes as compared to cryopreserved intact hepatocytes and intact enterocytes.

FIG. 20 shows CYP3A4 activity by permeabilized human enterocytes upon treatment by various concentrations of herbal supplements.

### DETAILED DESCRIPTION OF THE INVENTION

Provided herein are methods, compositions, and kits for evaluating xenobiotic metabolism, including both effects of the parent xenobiotic and its metabolites. In embodiments, the composition is an in vitro reagent that provides improved means for testing xenobiotic metabolism, such as a drug or drug candidate. In certain embodiments, the in vitro reagent is a cryopreserved mixture comprising metabolically competent cells (wherein cell membranes are permeabilized upon thawing;), exogenous drug metabolizing co-factors and a buffer solution such as cell culture medium, wherein the reagent does not comprise a cryopreservative agent. In embodiments, the in vitro reagent is frozen (e.g. cryopreserved) and comprises metabolically competent cells, exogenous drug metabolizing co-factors and cell culture medium, wherein the metabolically competent cells are permeabilized when thawed provided the in vitro reagent does not comprise a cryopreservative. In embodiments, the thawed in vitro reagent comprises metabolically competent cells with permeabilized plasma membranes, exogenous drug metabolizing co-factors and a buffer solution, wherein the metabolically competent cells are permeabilized via thawing. As used herein, cryopreserved refers to preserving a mixture of cells and enzyme co-factors (i.e. present in vitro reagent) that has been frozen at a temperature of about −10° C. or less, such as about −20° C. to about −80° C., in the absence of a cryopreservative agent.

The present in vitro reagent comprising permeabilized metabolically competent cells permeabilized via thawing of the cryopreserved intact metabolically competent cells provides benefits and advantages over use of organelle systems, intact cells and even metabolically competent cells permeabilized by other methods (e.g. sonication and/or detergent). Permeability induced by freezing without cryopreservative allows minimum disturbance to cellular integrity. Previous methods using a detergent (saponin) and/or extensive sonication cause total cell disruption, including disruption of the organelles. In contrast, we found permeability induced by freezing without cryopreservative allows the evaluation of drug metabolism in cells with near intact cell integrity but with permeable membrane; thereby the present in vitro reagent and methods are physiologically more relevant. In embodiments is provided an in vitro reagent comprising metabolically competent cells that are permeabilized upon thawing and supplemented with metabolic cofactors for drug metabolism studies and wherein the reagent does not comprise a cryopreservative agent. In embodiments, the present metabolically competent cells were not permeabilized via mechanical or detergent means.

The present in vitro reagent comprised of intact, permeabilized metabolically competent cells and exogenous drug metabolizing enzyme co-factors, represent a complete system with all the drug metabolizing enzymes as intact hepatocytes or intact enterocytes which is the major drawback of tissue homogenate systems such as S9 or microsomes (discussed in the Background section). The metabolically competent cells are permeabilized to allow supplementation of the in vitro reagent with exogenous metabolism enzyme cofactors to overcome the loss of cofactors due to cell isolation from the intact organ and the subsequent cryopreservation. The present in vitro reagent can be stored in a regular laboratory freezer at −10 or −80° C., without a need for liquid nitrogen freezers. In embodiments, the in vitro reagent can be thawed and use directly, without a need to supplement with cofactors, such as those needed for S9 and microsomes, and without a need for cell recovery/viability determination/cell density adjustment as needed when intact hepatocytes are used. The present in vitro reagent thereby has the advantage of the physiological relevance of, for example, intact hepatocytes and the ease of use of liver microsomes.

In embodiments, the composition is an exogenous metabolic system, wherein the system comprises: a) a cell culture medium admixture comprising; i) metabolically competent cells comprising permeabilized plasma membranes; and, ii) exogenous drug metabolizing enzyme co-factors, and b) metabolically incompetent target cells. The cell culture admixture is also referred to herein as the thawed in vitro reagent. In embodiments, the thawed in vitro reagent is added to a cell culture assay comprising metabolically incompetent target cells followed by addition of the test compound, incubation for a designated time duration, and quantification of the designated biological effects in the target cells, thereby allowing the evaluation of the effects of both the parent the metabolites.

In certain embodiments, the exogenous metabolic system comprises a separation between the cell culture admixture and the metabolically incompetent cells. The exogenous metabolic system may further comprise a cell culture vessel comprising one or more assay wells, wherein the assay wells comprise the cell culture medium admixture. In particular the metabolically incompetent target cells are in fluid communication with the cell culture medium admixture wherein, for example, each assay well is divided into a first and second chamber, wherein a first chamber comprises the cell culture medium admixture and the second chamber comprises the metabolically incompetent target cells provided the metabolically incompetent target cells are in fluid communication with the cell culture medium admixture.

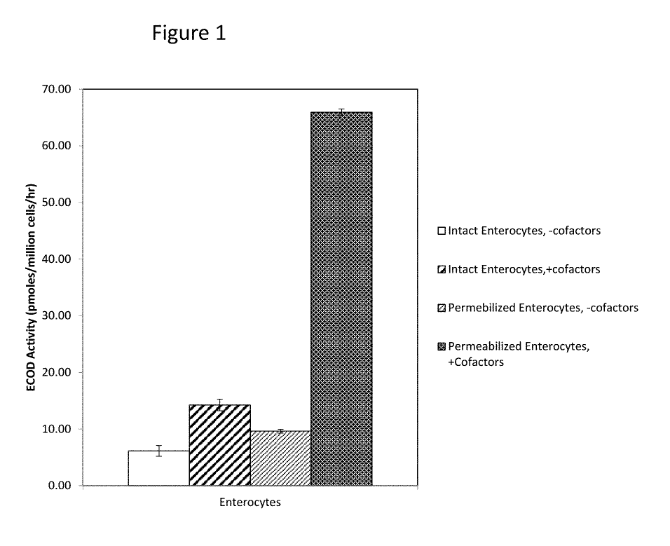

The present in vitro reagent, and its use in an exogenous metabolic system, provides advantages as compared to the use of intact metabolically competent cells or homogenates of those cells that require the use of enzyme co-factors. Those advantages, include but are not limited to, A) increased drug metabolizing enzyme activity; B) increased efficiency wherein the permeabilized metabolically competent cells admixed with exogenous drug metabolizing co-factors can be used directly, without centrifugation and cell counting (See FIG. 1 and Example 4); and C) improved access for researchers wherein the in vitro reagent can be stored in a freezer at −10° C. or −80° C., rather than in liquid nitrogen as needed for intact hepatocytes, allowing researchers without assess to liquid nitrogen storage systems use of the instant in vitro reagent.

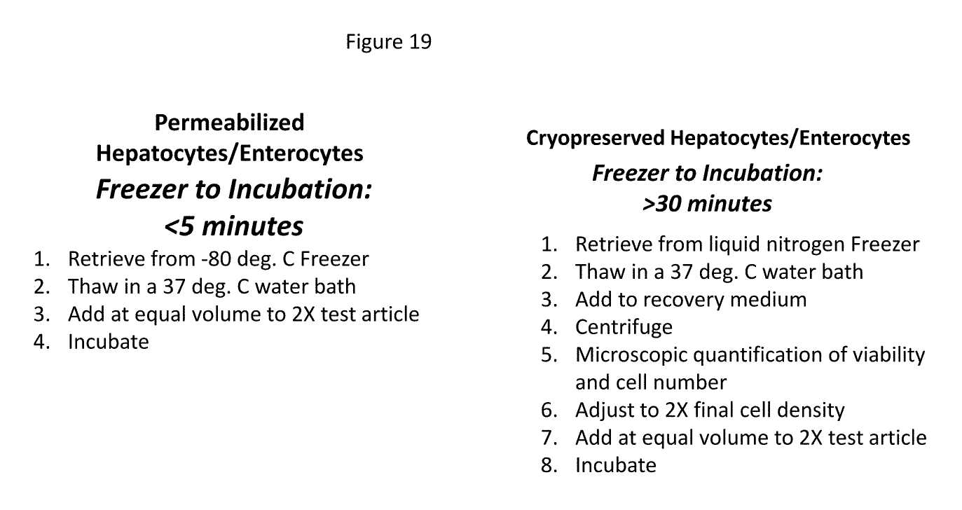

In embodiments, the metabolically competent cells are cryopreserved with the exogenous drug metabolizing co-factors and may be used directly to evaluate xenobiotic metabolism. In embodiments, the metabolically competent cells are permeabilized when they are cryopreserved in the absence of a cryoprotectant, which induces membrane permeability when the cells are thawed. Once thawed, the cells may be used directly without a step of cell counting or centrifugation. The amount of time from thawing to adding of a test compound may be from about 2 minutes to about 7 minutes, or less than about 6 minutes. See FIG. 19. In certain embodiments, the in vitro reagent does not comprise a cryopreservative agent. In other embodiments, the metabolically competent cells may be permeabilized prior to cryopreservation. In that instance, the in vitro reagent may comprise a cryopreservative agent.

In embodiments, the in vitro reagent comprises metabolically competent cells, exogenous drug metabolizing co-factors and a buffer solution such as a cell culture medium, wherein the co-factors are admixed with the competent cells in the cell culture medium and then the admixture is cryopreserved; also referred to herein as a cryopreserved mixture. In embodiments, the in vitro reagent is stored at a temperature of −10° C. or less, such as about −10° C. to about −80° C. In embodiments, the exogenous drug metabolizing co-factors include, but are not limited to those for oxidative metabolism, glucuronidation or sulfation. In embodiments, the in vitro reagent comprises, individually or in combination, β-nicotinamide adenine dinucleotide 2′-phosphate (NADPH) for oxidative metabolism, uridine 5′-diphosphoglucuronic acid (UDPGA) for glucuronidation and/or 3′-phosphoadenosine 5′-phosphosulfate (PAPS) for sulfation, N-acetyl coenzyme A, s-adenosyl methionine, amino acids, carnitine, and L-glutathione. See Example 1. As used herein, “drug” is used generically in the term “drug metabolizing enzyme co-factors” and is understood to broadly mean xenobiotic. In embodiments, the in vitro reagent comprises co-factors required for biochemical pathways and xenobiotic metabolism.

In embodiments, the manufacturing of the instant in vitro reagent is carried out by permeabilizing metabolically competent cells and adding exogenous drug metabolizing enzyme co-factors either before or after the metabolically competent cells are permeabilized, wherein the in vitro reagent is provided in a cell culture medium. In certain embodiments, the metabolically competent cells are frozen without a cryoprotectant (also referred to herein as a cryopreservative) wherein they are permeabilized during thaw of the in vitro reagent. In that instance, the metabolically competent cells (prior to being permeabilized) are admixed with the exogenous drug metabolizing co-factors in the presence of cell culture medium. As used in the art, a cryoprotectant or cryopreservative, is a substance that prevents damage to cells and/or organelles during freezing, and includes but is not limited to glycols; such as glycerol, ethylene glycol and propylene glycol; and dimethyl sulfoxide (DMSO). In embodiments, the in vitro reagent does not comprise glycerol and/or DMSO. In embodiments, the in vitro reagent is stored without serum, in other words the frozen in vitro reagent is serum free. In embodiments, the in vitro reagent is frozen at a temperature of −10° C. or less (e.g. from about −10° C. to about −80° C.), and does not comprise glycerol and/or DMSO and/or serum.

The methods of use herein are carried out by addition of a xenobiotic test compound or test article to the thawed in vitro reagent (admixture of metabolically competent cells with permeabilized plasma membranes and enzyme co-factors), i.e. room temperature (e.g. about 20 to 25° C.) to about a physiological temperature (e.g. about 35° C. to 40° C.), in a cell culture vessel. The test compound is introduced into the cell culture vessel comprising the thawed in vitro reagent, incubated, followed by evaluation of the effects of metabolism on the test compound including quantification of the parent compound for the evaluation of metabolic stability; metabolite quantification and identification; evaluation of drug metabolizing enzyme (e.g. P450) activity for evaluation of drug-drug interaction potential. The reagent is also used as an exogenous metabolic system in conjunction with a cell culture system comprising metabolically incompetent target cells.

In embodiments, a xenobiotic test compound or test article is added as an exogenous metabolic system, which comprises a thawed in vitro reagent (i.e. a cell culture medium admixture comprising metabolically competent cells comprising permeabilized plasma membranes and exogenous drug metabolizing enzyme co-factors) and metabolically incompetent target cells. In embodiments, the cell culture medium, the metabolically competent cells or the metabolically incompetent cells are subjected to the end point analysis to determine metabolism of the xenobiotic test compound and any effects of the metabolites thereof.

The test compounds used in the present invention include, but are not limited to drugs, drug candidates, biologicals, food components, herb or plant components, proteins, peptides, oligonucleotides, DNA and RNA. In embodiments, the test compound is a drug, a drug candidate, an industrial chemical, an environmental pollutant, a pesticide, an insecticide, a biological chemical, a vaccine preparation, a cytotoxic chemical, a mutagen, a hormone, an inhibitory compound, a chemotherapeutic agent or a chemical. In certain embodiments, the drug or drug candidate is selected from the group consisting of an organic compound, an inorganic compound, a hormone, a growth factor, a cytokine, a reception, an antibody, an enzyme, a peptide, an aptamer or a vaccine. The test compound can be either naturally-occurring or synthetic, and can be organic or inorganic. A person skilled in the art will recognize that the test compound can be added to the in vitro reagent present in the cell culture medium in an appropriate solvent or buffer.

In certain embodiments, the in vitro reagent and/or exogenous metabolic system is used for high-throughput screening to test the metabolic effects or response to a range of test compounds. In that instance, the in vitro reagent may be used with a cell culture vessel that is a multi-well plate, such as a 6-well; 12-well; 24-well; 48-well, 96-well; 384-well, 1536-well plate or any combination thereof. In alternative embodiments, the methods use a cell culture vessel with a single assay well.

In embodiments, the metabolic system comprises a cell culture vessel wherein the metabolically incompetent target cells are in fluid communication with the cell culture medium admixture. In embodiments, the cell culture vessel comprises one or more assay wells, wherein the assay wells comprise the cell culture medium admixture and the metabolically incompetent cells in the same admixture. In alternative embodiments, each assay well is divided into a first and second chamber, wherein a first chamber comprises the cell culture medium admixture and the second chamber comprises the metabolically incompetent target cells provided the metabolically incompetent target cells are in fluid communication with the cell culture medium admixture. In certain embodiments, the first and second chamber are separated by a porous membrane that is liquid-permeable and cell-impermeable.

In embodiments, the exogenous metabolic system comprises a cell culture vessel with a single well plate. Alternatively, the exogenous metabolic system comprises cell culture vessel that is a multi-well plate. In embodiments, the porous membrane separating the first and second chamber of the cell culture vessel comprises polyethylene terephthalate (PET), polycarbonate, polystyrene, polypropylene, nylon, Mylar, stainless steel, wire mesh, aluminum, synthetic mesh, plastic, or paper. In embodiments, the cell culture insert is a transwell.

In embodiments, the metabolically competent cells are primary cells or cell lines; and can be human, rat, monkey, dog, mammals, avian or non-mammalian cells. In certain embodiments, the metabolically competent cells are from organs responsible for drug metabolism in vivo, such as liver (hepatocytes), intestine (enterocytes) and kidney (renal cells). In certain embodiments, the metabolically competent cells are human hepatocytes. See, Li, A. P. (2007) Human hepatocytes: isolation, cryopreservation and applications in drug development. Chemico-biological interactions, 168(1), 16-29. Freshly prepared metabolically competent cells or cryopreserved cells (e.g. hepatocytes) obtained by standard methods from a human or animal liver can be used as the metabolically competent cells. See, Hewitt, Nicola J., et al. “Primary hepatocytes: current understanding of the regulation of metabolic enzymes and transporter proteins, and pharmaceutical practice for the use of hepatocytes in metabolism, enzyme induction, transport, clearance, and hepatotoxicity studies.” Drug metabolism reviews 39.1 (2007): 159-234.

In embodiments, the metabolically competent cells are hepatocytes (the parenchymal cells of the liver). Hepatic metabolism is known to be the major determinant of metabolism-dependent xenobiotic toxicity. P450 and non-P450 phase 1 oxidation enzyme pathways are responsible mostly for the bioactivation of relatively inert parent compounds to reactive (toxic/carcinogenic/mutagenic) metabolites. Phase 2 conjugating pathways are responsible mostly for the biotransformation of toxic parent compounds or metabolites to less toxic compounds. Both phase 1 and phase 2 pathways are present in the hepatocytes—the key hepatic cell type responsible for hepatic metabolism. In embodiments, enterocytes can be used to model enteric metabolism for orally ingested toxicants. In other embodiments, renal proximal tubule cells can be used to model renal metabolism. In certain other embodiments, enterocytes can be used to model intestinal metabolism.

In certain embodiments, the metabolically competent cells are recombinant cells, wherein for example, they have been engineered to comprise drug metabolizing enzymes. In certain embodiments, the metabolically competent cells are engineered to contain cytochrome P450 isoforms. See, Doehmer, J., Battula, N., Wölfel, C., Kudla, C., Keita, Y. and Staib, A. H., 1992. Biotransformation of caffeine and theophylline in mammalian cell lines genetically engineered for expression of single cytochrome P450 isoforms. Biochemical pharmacology, 43(2), pp. 225-235; Donato, M. T., Jiménez, N., Castell, J. V., & Gómez-Lechón, M. J. (2004). Fluorescence-based assays for screening nine cytochrome P450 (P450) activities in intact cells expressing individual human P450 enzymes. Drug Metabolism and Disposition, 32(7), 699-706.

In embodiments, a stable cell line such as HepG2 is used in the present in vitro reagent and methods for determining metabolism of a test compound. HepG2 is a perpetual cell line derived from the liver of a 15-year-old Caucasian male with a well-differentiated hepatocellular carcinoma. Because of the high degree of morphological and functional differentiation in vitro, HepG2 cells can be a suitable model to study the intracellular trafficking and dynamics of bile canalicular and sinusoidal membrane proteins and lipids in human hepatocytes in vitro. See, Ihrke et al., WIF-B cells: an in vitro model for studies of hepatocyte polarity. Journal of Cell Biology 123 (6), 1761-1775, 1993. In certain embodiments, a stable cell line such as HepaRG is used in the present in vitro reagent and methods for determining metabolism of a test compound. See, Guillouzo, A., Corlu, A., Aninat, C., Glaise, D., Morel, F. and Guguen-Guillouzo, C., 2007. The human hepatoma HepaRG cells: a highly differentiated model for studies of liver metabolism and toxicity of xenobiotics. Chemico-biological interactions, 168(1), pp. 66-73.

In embodiments, present metabolically competent cells are genetically altered or modified so as to contain a non-native “recombinant” (also called “exogenous”) nucleic acid sequence, or modified by antisense technology to provide a gain or loss of genetic function. As used herein, “cells”, unless specified otherwise, refers to metabolically competent cells. Methods for generating genetically modified cells are known in the art, see for example “Current Protocols in Molecular Biology,” Ausubel et al., eds, John Wiley & Sons, New York, N.Y., 2000.

Accordingly, in embodiments, the present in vitro reagent may comprise any cell type, including primary cells, stem cells, progenitor cells, normal, genetically-modified, genetically altered, immortalized, and transformed cell lines, provided the cells are metabolically competent, permeabilized (permeabilized during thaw), and admixed with exogenous drug metabolizing enzyme co-factors. The present invention is suitable for use with single cell types or cell lines, or with combinations of different cell types. Preferably the cultured cells maintain the ability to respond to stimuli that elicit a response in their naturally occurring counterparts. These may be derived from all sources such as eukaryotic (mammalian and non-mammalian). The eukaryotic cells can be animal in nature, such as human, simian, or rodent. They may be of any tissue type (e.g., heart, stomach, kidney, intestine, lung, liver, fat, bone, cartilage, skeletal muscle, smooth muscle, cardiac muscle, bone marrow, muscle, brain, pancreas), and cell type (e.g., epithelial, endothelial, mesenchymal, adipocyte, hematopoietic).

In embodiments, metabolically competent cells are cultured from a variety of genetically diverse individuals who may respond differently to biologic and pharmacologic agents. Genetic diversity can have indirect and direct effects on metabolism of a test compound. In embodiments, the metabolically competent cells are a pool of cells from multiple individuals or donors. In certain embodiments, the metabolically competent cells are reflective of the heterogeneity of a population of individuals.

In embodiments, an exogenous metabolic system comprises metabolically incompetent cells (in addition to a thawed in vitro reagent wherein the metabolically competent cells have a permeabilized plasma membrane). In embodiments, the metabolically incompetent cells are mammalian or non-mammalian and include, but are not limited to carbiomyocytes (Doherty, Kimberly R., Robert L. Wappel, Dominique R. Talbert, Patricia B. Trusk, Diarmuid M. Moran, James W. Kramer, Arthur M. Brown, Scott A. Shell, and Sarah Bacus. “Multi-parameter in vitro toxicity testing of crizotinib, sunitinib, erlotinib, and nilotinib in human cardiomyocytes.” Toxicology and applied pharmacology 272, no. 1 (2013): 245-255), vascular endothelia cells (Louise, C. B. and Obrig, T. G., 1991. Shiga toxin-associated hemolytic-uremic syndrome: combined cytotoxic effects of Shiga toxin, interleukin-1 beta, and tumor necrosis factor alpha on human vascular endothelial cells in vitro. Infection and immunity, 59(11), pp. 4173-4179) and cell lines such as mouse 3T3 fibroblasts (Spielmann, H., Balls, M., Brand, M., Döring, B., Holzhütter, H. G., Kalweit, S., Klecak, G., Eplattenier, H. L., Liebsch, M., Lovell, W. W. and Maurer, T., 1994. EEC/COLIPA project on in vitro phototoxicity testing: First results obtained with a Balb/c 3T3 cell phototoxicity assay. Toxicology in vitro, 8(4), pp. 793-796) and Chinese hamster ovary cells (Li, A. P., Carver, J. H., Choy, W. N., Hsie, A. W., Gupta, R. S., Loveday, K. S., O'neill, J. P., Riddle, J. C., Stankowski, L. F. and Yang, L. L., 1987. A guide for the performance of the Chinese hamster ovary cell/hypoxanthine-guanine phosphoribosyl transferase gene mutation assay. Mutation Research/Genetic Toxicology, 189(2), pp. 135-141).

In embodiment, examples include reporter cell lines for endocrine disruption, but are not limited to metabolically incompetent cells disclosed in the following references: Willemsen, P., Scippo, M. L., Kausel, G., Figueroa, J., Maghuin-Rogister, G., Martial, J. A. and Muller, M., 2004. Use of reporter cell lines for detection of endocrine-disrupter activity. Analytical and bioanalytical chemistry, 378(3), pp. 655-663), Genotoxicity determination with the Ames salmonella typhimurium gene mutation assay; De Flora, S., Zanacchi, P., Camoirano, A., Bennicelli, C. and Badolati, G. S., 1984. Genotoxic activity and potency of 135 compounds in the Ames reversion test and in a bacterial DNA-repair test. Mutation Research/Reviews in Genetic Toxicology, 133(3), pp. 161-198.

In embodiments, examples of metabolically incompetent cells include mammalian cell lines used for genotoxicity assays, but are not limited to metabolically incompetent cells disclosed in the following references: Moore, Martha M., et al. “Mouse lymphoma thymidine kinase gene mutation assay: Follow-up meeting of the international workshop on Genotoxicity testing—Aberdeen, Scotland, 2003—Assay acceptance criteria, positive controls, and data evaluation.” Environmental and molecular mutagenesis 47.1 (2006): 1-5; Aaron, C. S., Bolcsfoldi, G., Glatt, H. R., Moore, M., Nishi, Y., Stankowski, L., Theiss, J. and Thompson, E., 1994. Mammalian cell gene mutation assays working group report. Mutation Research/Environmental Mutagenesis and Related Subjects, 312(3), pp. 235-239.).

In embodiments, examples of metabolically incompetent cells include nonhepatic primary cells for the evaluation of organ-specific drug toxicity, but are not limited to metabolically incompetent cells disclosed in the following references: Pfaller, W. and Gstraunthaler, G., 1998. Nephrotoxicity testing in vitro—what we know and what we need to know. Environmental health perspectives, 106(Suppl 2), p. 559; Mordwinkin, N. M., Burridge, P. W. and Wu, J. C., 2013. A review of human pluripotent stem cell-derived cardiomyocytes for high-throughput drug discovery, cardiotoxicity screening, and publication standards. Journal of cardiovascular translational research, 6(1), pp. 22-30).

For screening test compounds using the in vitro reagent and/or exogenous metabolic system for their metabolic effect on the cells, the in vitro reagent comprising metabolically competent cells and exogenous drug metabolizing enzyme co-factors are thawed and placed in a cell culture vessel with cell culture medium. If using the exogenous metabolic system, the system further comprises metabolically in competent cells in fluid communication with the thawed in vitro reagent. The term “culture condition” encompasses cells, media, factors, time and temperature, atmospheric conditions, pH, salt composition, minerals, etc. Cell culturing is typically performed in a sterile environment mimicking physiological conditions, for example, at 37° C. in an incubator containing a humidified 92-95% air/5-8% CO₂ atmosphere. In embodiments, the cell culture temperate is a range from 33-40° C. Cell culturing may be carried out in nutrient mixtures containing undefined biological fluids such a fetal calf serum, or media that is fully defined and serum free. A variety of culture media are known in the art and are commercially available.

In embodiments, the metabolically competent cells (with permeabilized plasma membrane) are not checked for viability, centrifuged, counted or cultured prior to addition of the xenobiotic test compound. In alternative embodiments, the in vitro reagent may be cultured for a time period from a few hours to days prior to use in the instant methods. In embodiments, the xenobiotic test compound is placed in the cell culture vessel wherein the thawed in vitro reagent (i.e. cell culture medium admixture comprising permeabilized metabolically competent cells and exogenous drug metabolizing enzyme co-factors) is then incubated under appropriate cell culture conditions as disclosed herein for a time period of 30 minutes to up to 10 days. In embodiments, the incubation period can be at least 1 hours, 2 hours, 5 hours, 10 hours, 15 hours, 24 hours, 2 days, 3 days, 4 days, 5 days, 6 days, 7 days, 8 days, 9 days or at least 10 days. In embodiments, the incubation time period is not longer than 1 day, 2 days, 5 days, 6 days, 7 days, 8 days, 9 days or not longer than 10 days. In embodiments, the cell culture conditions replicate physiological conditions as much as possible. The term “physiological conditions” as used herein is defined to mean that the cell culturing conditions are very specifically monitored to mimic as closely as possible the natural tissue conditions for a particular type of cell in vivo.

In embodiments, the xenobiotic test compound is considered an input variable, and is used interchangeably herein with a test compound. The test compounds are screened for biological activity by adding to a pharmacokinetic-based culture system (e.g. present in vitro reagent), and then assessing the metabolically competent cells (or culture medium) for changes in output variables of interest, e.g., consumption of O₂, production of CO₂, cell viability, expression of proteins of interest (protein expression), cell function, expression of genes of interest (gene expression), metabolite formation or metabolite profiles. The test compound is typically added in solution, or readily soluble form, to the medium of cells in culture. The test compound can be added using a flow through system, or alternatively, adding a bolus to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall composition of the culture medium should not change significantly with the addition of the bolus, or between the two solutions in a flow through method.

In embodiments, the test compound includes pharmacologically active drugs or drug candidates and genetically active molecules. Test compounds of interest include chemotherapeutic agents, anti-inflammatory agents, hormones or hormone antagonists, ion channel modifiers, and neuroactive agents. Exemplary of pharmaceutical agents suitable for this invention are those described in “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming Organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

In embodiments, the test compound includes all of the classes of molecules disclosed herein, and may further or separately comprise samples of unknown content. While many samples will comprise compounds in solution, solid samples that can be dissolved in a suitable solvent may also be assayed. Samples containing test compounds of interest include environmental samples, e.g., ground water, sea water, or mining waste; biological samples, e.g., lysates prepared from crops or tissue samples; manufacturing samples, e.g., time course during preparation of pharmaceuticals; as well as libraries of compounds prepared for analysis; and the like. Samples of interest include test compounds being assessed for potential therapeutic value, e.g., drug candidates from plant or fungal cells.

Test compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, naturally or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification to produce structural analogs.

In embodiments, following incubation of the thawed in vitro reagent with the test compound an end point analysis is performed to determine the effect of the test compound on the metabolically competent cells. In embodiments, the end point analysis identifies the output variable (e.g. the effect of the test compound) of the in vitro reagent. In embodiments, output variables are quantifiable elements of the cells, particularly elements that can be accurately measured in a cell culture system. An output variable can be any cell component or cell product including, e.g., viability, respiration, metabolism, cell surface determinant, receptor, protein or conformational or posttranslational modification thereof, lipid, carbohydrate, organic or inorganic molecule, mRNA, DNA, or a portion derived from such a cell component. In embodiments, the output variable is directly or indirectly a result of the test compound or its metabolite. While most output variables will provide a quantitative readout, in some instances a semi-quantitative or qualitative result will be obtained. Readouts may include a single determined value, or may include mean, median value or the variance. Characteristically a range of readout values will be obtained for each output. Variability is expected and a range of values for a set of test outputs can be established using standard statistical methods.

In embodiments, the end point assay is an Ames Salmonella histidine reversion assay (Ames test) for genotoxicity, a mammalian or non-mammalian genotoxicity assay, a mammalian or non-mammalian pharmacological assay.

Various methods can be utilized for quantifying the presence of selected metabolism markers. Liquid chromatography (LC), mass spectrometry (MS), and their combination (LC/MS-MS) are routinely used for the quantification of metabolites. For non-LC/MS measurement of the amount of a molecule that is present, a convenient method is to label the molecule with a detectable moiety, which may be fluorescent, luminescent, radioactive, or enzymatically active. Fluorescent and luminescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g., by expressing them as green fluorescent protein chimeras inside cells (for a review, See Jones et al. (1999) Trends Biotechnol. 17(12):477-81).

Output variables may be measured by immunoassay techniques such as, immunohistochemistry, radioimmunoassay (RIA) or enzyme linked immunosorbance assay (ELISA) and related non-enzymatic techniques. These techniques utilize specific antibodies as reporter molecules that are particularly useful due to their high degree of specificity for attaching to a single molecular target. Cell based ELISA or related non-enzymatic or fluorescence-based methods enable measurement of cell surface parameters. Readouts from such assays may be the mean fluorescence associated with individual fluorescent antibody-detected cell surface molecules or cytokines, or the average fluorescence intensity, the median fluorescence intensity, the variance in fluorescence intensity, or some relationship among these. For toxicity assays, outputs can include measurement of cell viability such as enzyme release, cellular ATP contents, reactive oxygen species formation, decrease of reduced glutathione, protein synthesis, protein contents, DNA contents, dye exclusion, dye inclusion, and cell detachment. For pharmacological assays, specific disease target related assays can be used. For genotoxicity assays, endpoints measured may include DNA damage, chromosomal aberration, mutant generation, and induction of DNA repair.

In embodiments, the results of screening assays may be compared to results obtained from a reference compound, concentration curves, controls (with and without metabolically competent cells), etc. The comparison of results is accomplished by the use of suitable deduction protocols, A1 systems, statistical comparisons, etc.

A database of reference output data can be compiled. These databases may include results from known agents or combinations of agents, as well as references from the analysis of cells treated under environmental conditions in which single or multiple environmental conditions or parameters are removed or specifically altered. A data matrix may be generated, where each point of the data matrix corresponds to a readout from an output variable, where data for each output may come from replicate determinations, e.g., multiple individual cells of the same type.

The readout may be a mean, average, median or the variance or other statistically or mathematically derived value associated with the measurement. The output readout information may be further refined by direct comparison with the corresponding reference readout. The absolute values obtained for each output under identical conditions will display a variability that is inherent in live biological systems and also reflects individual cellular variability as well as the variability inherent between individuals.

### Exemplary Embodiments

In illustrative embodiments, the in vitro reagent is a cryopreserved mixture comprising metabolically competent cells (cells wherein cell membranes are permeabilized upon thawing), exogenous drug metabolizing enzyme co-factors and a buffered solution such as cell culture medium, wherein the reagent does not comprise a cryopreservative agent. The xenobiotic test article or compound (e.g. drug candidate) is metabolized by the metabolically competent cells in the presence of the exogenous co-factors.

In exemplary embodiments the present in vitro reagent is prepared for use to evaluate the metabolic effects of a test compound using cryopreserved metabolically competent cells with a permeabilized cell membrane, comprising the following steps: A) permeabilizing metabolically competent cells (e.g. hepatocytes or enterocytes); B) adding exogenous drug metabolizing enzyme co-factors either before or after the metabolically competent cells are permeabilized (e.g. NADPH, UDPGA, and PAPS); C) providing a cell culture medium. In embodiments, the in vitro reagent is further stored at a temperature of −10° C. or lower. In illustrative embodiments, the cell membrane of the metabolically competent cells is permeabilized when thawed.

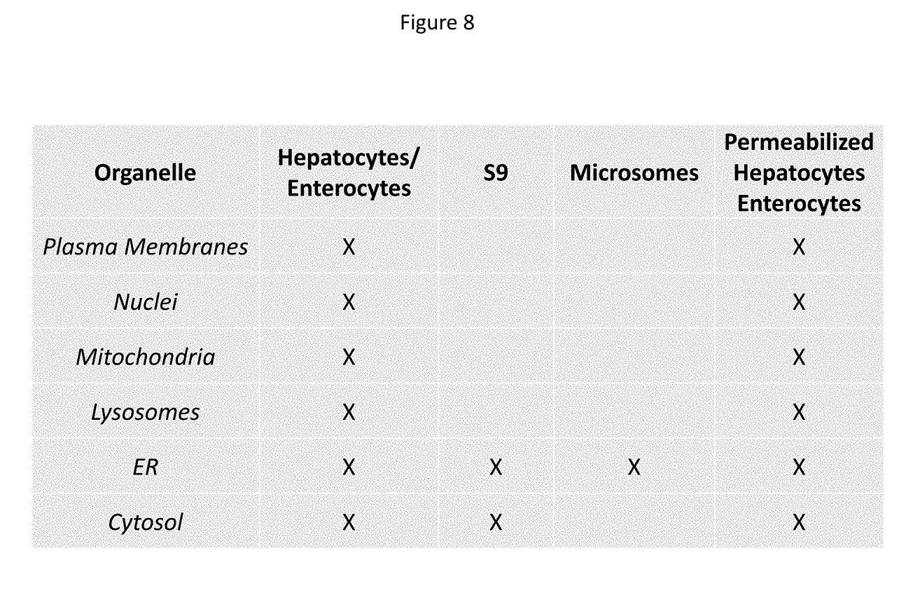

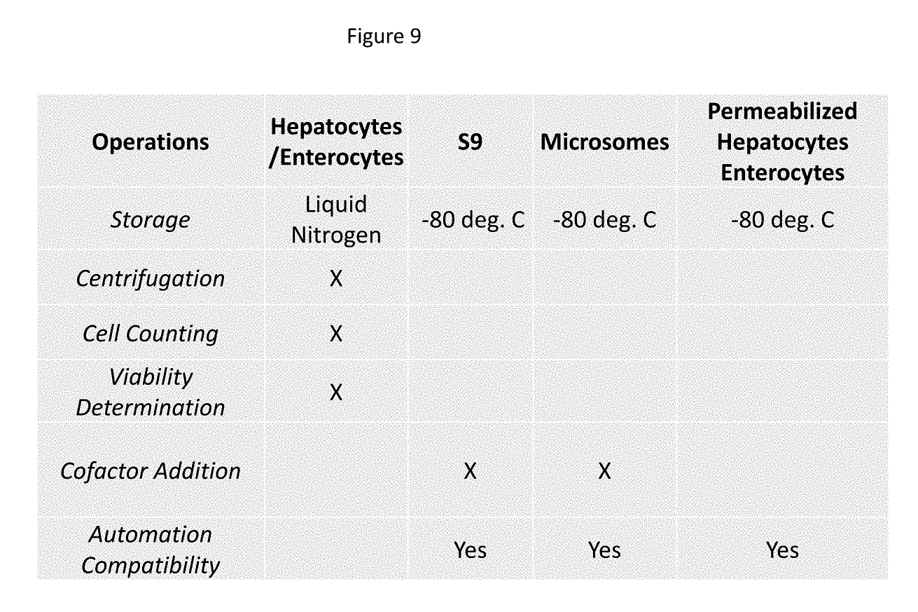

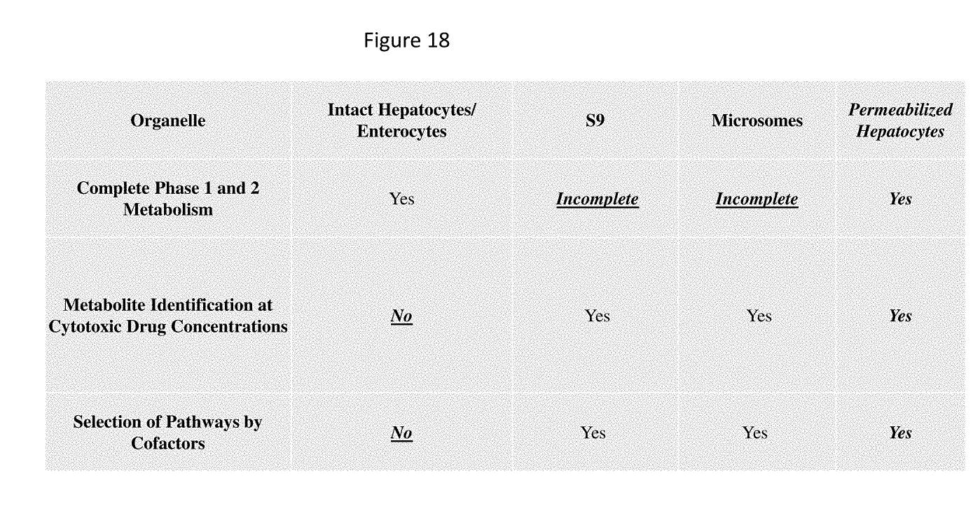

In exemplary embodiments, the present in vitro reagent is used to evaluate the metabolic effects of a xenobiotic test compound comprising the following steps: A) thawing the in vitro reagent (prepared as disclosed herein) and placing in a cell culture vessel; B) introducing the xenobiotic test compound into the cell culture vessel; C) incubating the test compound for 0.5 h to 10 days at 33-40° C.; and; D) performing an end point assay of the in vitro reagent or cell culture medium to determine metabolism of the xenobiotic test compound. See Examples 1-15 and FIGS. 1-20.

### EXAMPLES

The following examples are put forth so as to provide those of ordinary skill in the art with a complete disclosure and description of how to use the embodiments provided herein, and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by volume, and temperature is in degrees Centigrade. It should be understood that variations in the methods as described can be made without changing the fundamental aspects that the Examples are meant to illustrate.

### Example 1: In Vitro Reagent Containing Permeabilized Hepatocytes for the Evaluation of a Xenobiotic Test Compound

Hepatocytes are isolated from human livers by collagenase digestion. The hepatocytes may be utilized from a single donor, or pooled from multiple donors. The hepatocytes are then cryopreserved in a serum free cell culture medium (Hepatocyte Incubation Medium (HQM), In Vitro ADMET Laboratories Inc., Columbia, Md.)) in the presence of drug metabolizing enzyme cofactors: 2 mM NADPH, 5 mM UDPGA, and 1 mM PAPS at a concentration of 1 million cells per mL. The cells are aliquoted at 5 mL aliquots and frozen in a −80 deg. C. freezer. On the day of use, the hepatocyte/cofactor mixture (i.e. in vitro reagent) is thawed at 4° C. and 50 μL of the mixture is added to 50 μL of HQM containing a xenobiotic test article at an appropriate concentration in wells of a 96-well plate. The plate is incubated in a cell culture incubator maintained at 37° C. for a specific time period (e.g. 60 minutes) after which an organic solvent (e.g. acetonitrile) as added at a volume of 100 μL to each well. The contents of the wells are collected for LC/MS-M S quantification of drug metabolism (e.g. parent disappearance; metabolite formation).

In embodiments, the hepatocytes are isolated from human livers by collagenase digestion and pooled from multiple donors to manufacture the present in vitro reagent containing permeabilized hepatocytes.

### Example 2: In Vitro Reagent Containing Permeabilized Enterocytes for the Evaluation of a Xenobiotic Test Compound

The assay was performed essentially as disclosed in Example 1 except enterocytes are isolated from human intestines by collagenase digestion. The enterocytes may be utilized from a single donor, or pooled from multiple donors. The enterocytes are then cryopreserved in a serum free cell culture medium in the presence of drug metabolizing enzyme cofactors: 2 mM NADPH, 5 mM UDPGA, and 1 mM PAPS at a concentration of 1 million cells per mL. The cells are aliquoted at 5 mL aliquots and frozen in a −80 deg. C. freezer. On the day of use, the enterocyte/cofactor mixture (i.e. in vitro reagent) is thawed at 4° C. and 50 μL of the mixture is added to 50 μL of HQM containing a xenobiotic test article at an appropriate concentration in wells of a 96-well plate. The plate is incubated in a cell culture incubator maintained at 37° C. for a specific time period (e.g. 60 minutes) after which an organic solvent (e.g. acetonitrile) as added at a volume of 100 μL to each well. The contents of the wells are collected for LC/MS-MS quantification of drug metabolism (e.g. parent disappearance; metabolite formation).

In embodiments, the enterocytes are isolated from human intestines by collagenase digestion and pooled from multiple donors to manufacture the present in vitro reagent containing permeabilized enterocytes.

TABLE 1
Drug Metabolism Enzyme Activities of Human Enterocytes

| Donor | CYP2C9 | CYP2C19 | CYP3A4 | UGT | SULT | 2J2 | CES2 |
|---|---|---|---|---|---|---|---|
| HE3005 | 1.68 | 0.56 | 2.7 | 8.38 | 8.72 | 1.20 | 0.23 |
| HE3006 | 0.59 | 0.29 | 0.13 | 2.30 | 2.04 | 0.73 | 0.33 |
| HE3007 | 0.91 | 0.39 | 0.99 | 3.08 | 4.04 | 0.57 | 0.46 |
| HE3008 | 0.46 | 0.68 | 0.87 | 1.80 | 1.79 | 0.33 | 0.55 |
| HE3009 | 1.18 | 0.35 | 0.72 | 4.32 | 7.78 | 0.25 | 0.60 |
| HE3010 | 1.21 | 0.62 | 0.46 | 2.56 | 3.32 | 0.99 | 0.41 |
| HE3011 | 0.03 | 0.01 | 0.09 | 1.01 | 1.70 | 0.33 | 0.29 |
| HE3013 | NA | NA | 0.2 | NA | NA | NA | NA |
| HE3014 | 0.44 | 0.11 | 0.4 | 3.55 | 2.66 | 1.18 | 0.17 |
| HE3015 | 2.50 | 0.49 | 2.55 | 7.33 | 5.23 | 0.95 | 0.51 |
| HE3016 | 2.05 | 1.08 | 1.0 | 5.71 | 4.13 | 0.93 | 0.34 |
| HE3019 | 0.24 | 0.11 | 0.30 | 1.47 | 1.89 | 0.26 | 0.30 |
| HE3020 | 0.31 | 0.14 | 0.5 | 5.83 | 1.84 | 0.58 | 0.59 |
| HE3021 | 0.20 | 0.06 | 0.17 | 1.49 | 1.64 | 0.19 | 0.08 |
| HE3027 | 2.02 | 0.31 | 0.7 | 3.68 | 2.69 | 0.76 | 0.19 |
| HE3028 | 0.68 | 0.21 | 0.82 | 3.84 | 9.02 | 0.71 | 0.30 |
| HE3029 | 0.86 | 0.12 | 0.6 | 6.55 | 3.65 | 0.76 | 0.08 |
| HE3031 | 0.34 | 0.09 | 0.16 | 1.60 | 0.79 | 0.49 | 0.18 |

### Example 3: In Vitro Reagent Containing Permeabilized Hepatocyte Cell Line for the Evaluation of a Xenobiotic Test Compound

The assay was performed essentially as disclosed in Example 1, except a hepatocyte cell line (e.g. HepaRG) known to contain drug metabolizing enzyme activity is used instead of primary hepatocytes. The hepatocyte cell line is cryopreserved in a serum free cell culture medium (Hepatocyte Incubation Medium (HQM), In Vitro ADMET Laboratories Inc., Columbia, Md.) in the presence of drug metabolizing enzyme cofactors: 2 mM NADPH, 5 mM UDPGA, and 1 mM PAPS at a concentration of I million cells per mL. The cells are aliquoted at 5 mL aliquots and frozen in a −80 deg. C. freezer. On the day of use, the hepatocyte/cofactor mixture (i.e. in vitro reagent) is thawed at 4° C. and 50 μL of the mixture is added to 50 μL. of HQM containing a xenobiotic test article at an appropriate concentration in wells of a 96-well plate. The plate is incubated in a cell culture incubator maintained at 37° C. for a specific time period (e.g. 60 minutes) after which an organic solvent (e.g. acetonitrile) as added at a volume of 100 uL to each well. The contents of the wells are collected for LC/MS-MS quantification of drug metabolism (e.g. parent disappearance; metabolite formation).

### Example 4: Comparison of the In Vitro Reagent as Compared to Intact (Non-Permeabilized) Metabolically Competent Cells (with or without Enzyme Co-Factor Added to the Intact Cells)

The assay was performed essentially as disclosed in Example 2, wherein the in vitro reagent comprises enterocytes and exogenous drug metabolizing enzyme co-factors (NADPH, UPGA, and PAPS), herein referred to as “cofactors”. As comparators, intact enterocytes, that were not permeabilized, were used wherein one group was incubated with cofactors and another group was incubated without cofactors, each in the presence of the xenobiotic test compound. Permeabilized enterocytes incubated without cofactors were used as a control. The test groups were: Group A—intact enterocytes incubated without cofactors; Group B—intact enterocytes incubated with cofactors; Group C—permeabilized enterocytes incubated without cofactors (control); Group D—the in vitro reagent (permeabilized enterocytes incubated with cofactors). Metabolism of 7-ethoxycoumarin (7-ethoxycoumarin O-deethylation (ECOD) activity) was used as a test xenobiotic compound to demonstrate the difference in metabolism of each test group. Highest activity was observed with permeabilized enterocytes with cofactors (+cofactors) Group D, demonstrating the advantage of the present in vitro reagent. Permeabilization (by freezing without cryoprotectants followed by thawing) allows maximal interaction of the intracellular enzymes with the substrate (7-ethoxycoumarin), and the addition of cofactors overcomes the dilution of intracellular cofactors due to permeabilization. The results demonstrate permeabilization and cofactor supplementation provides significantly higher enteric xenobiotic metabolism. See FIG. 1.

### Example 5: Evaluation and Detection of Key Drug Metabolizing Enzyme Activity

The assay was performed essentially as disclosed in Example 1. The cryopreserved hepatocyte/cofactor mixture (i.e. in vitro reagent) was thawed at 37° C. and 50 μL of the mixture is added to 50 μL of HQM containing metabolic enzyme substrates at an appropriate concentration in wells of a 96-well plate. The plate was incubated in a cell culture incubator maintained at 37° C. for a specific time period (e.g. 60 minutes) after which an organic solvent (e.g. acetonitrile) was added at a volume of 100 μL to each well. The contents of the wells are collected for LC/MS-MS quantification of metabolite formation.

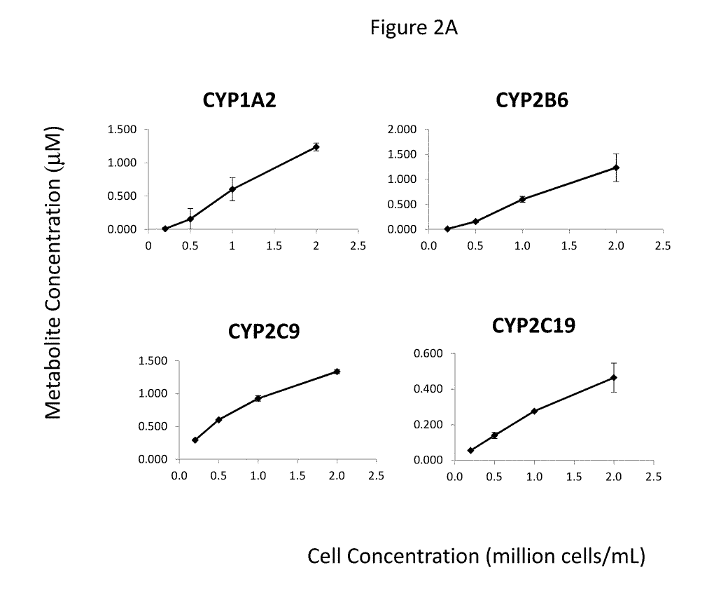

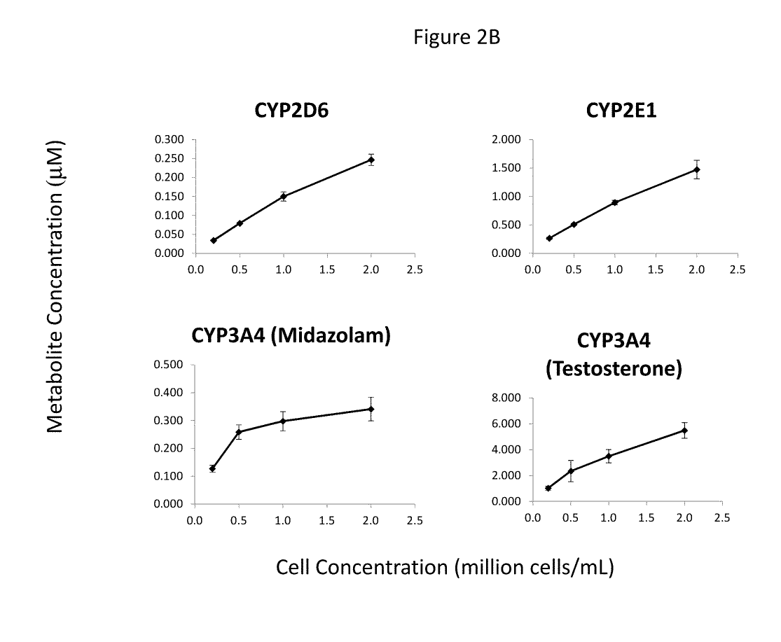

The results demonstrated the permeabilized human hepatocytes possessed all key drug metabolizing enzyme activities. The major P450 isoform activities as a function of cell concentrations are shown. The linear concentration versus activity curves demonstrate that permeabilized hepatocytes can be used routinely as an in vitro drug metabolism system for scientific investigations. See FIG. 2.

### Example 6: Evaluation and Detection of P450 Isoform-Selective Substrate in Intact Human Hepatocytes and the Present Permeabilized Hepatocytes Over a Period of 30 Minutes to 240 Minutes

The assay was performed essentially as disclosed in Example 1. The cryopreserved permeabilized hepatocytes/cofactor mixture was thawed at 37° C. and 50 μL of the mixture was added to 50 μL of HQM containing metabolic enzyme substrates at an appropriate concentration in wells of a 96-well plate. The plate was incubated in a cell culture incubator maintained at 37° C. for 30, 60, 120 and 240 minutes after which an organic solvent (e.g. acetonitrile) was added at a volume of 100 μL, to each well. The contents of the wells were collected for LC/MS-MS quantification of metabolite formation. The intact hepatocytes were thawed at 37° C., recovered by adding the thawed hepatocytes to UCRM, centrifuged at 100×g for 10 minutes, resuspended in HQM followed by microscopic evaluation of viability based on trypan blue, cell concentration adjusted with HQM to 2 million cells per mL, followed by incubation with drug metabolism substrates as described above for the permeabilized hepatocytes.

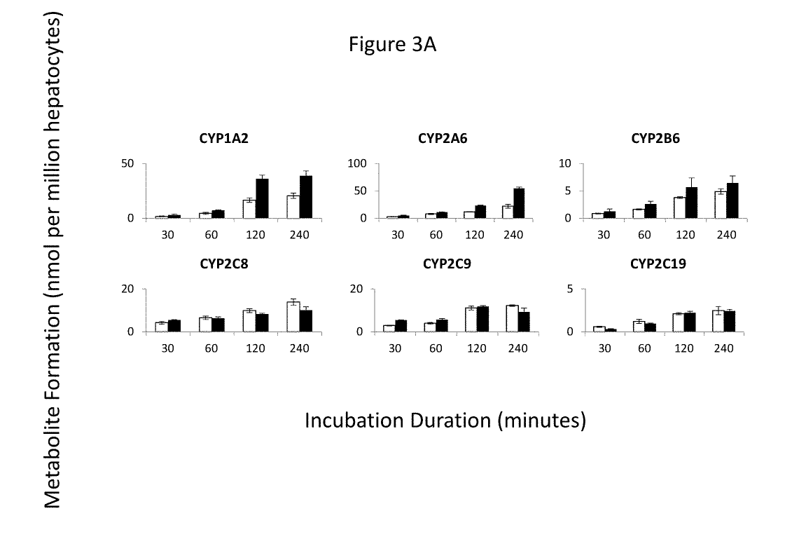

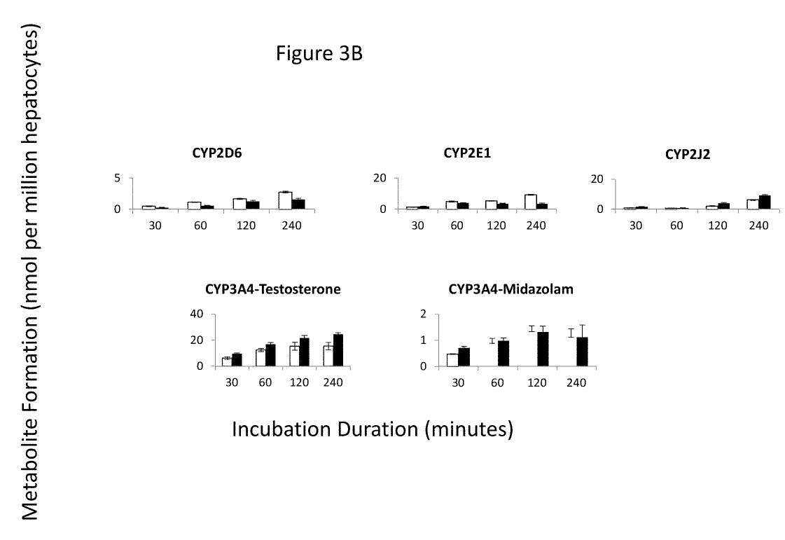

The results demonstrate a similar trend in the linear time course of metabolite formation in both the intact human hepatocytes and the present permeabilized hepatocytes. See FIG. 3

### Example 7: Evaluation and Detection of Non-P450 Pathway-Selective Substrate in Intact Human Hepatocytes and the Present Permeabilized Hepatocytes Over a Period of 30 Minutes to 240 Minutes

The assay was performed essentially as disclosed in Example 1. The cryopreserved permeabilized hepatocytes/cofactor mixture was thawed at 37° C. and 50 μL of the mixture is added to 50 μL of HQM containing metabolic enzyme substrates at an appropriate concentration in wells of a 96-well plate. The plate was incubated in a cell culture incubator maintained at 37° C. for 30, 60, 120 and 240 minutes after which an organic solvent (e.g. acetonitrile) was added at a volume of 100 μL to each well. The contents of the wells are collected for LC/MS-MS quantification of metabolite formation. The intact hepatocytes were thawed at 37° C., recovered by adding the thawed hepatocytes to UCRM, centrifuged at 100×g for 10 minutes, resuspended in HQM followed by microscopic evaluation of viability based on trypan blue, cell concentration adjusted with HQM to 2 million cells per mL, followed by incubation with drug metabolism substrates as described above for the permeabilized hepatocytes.

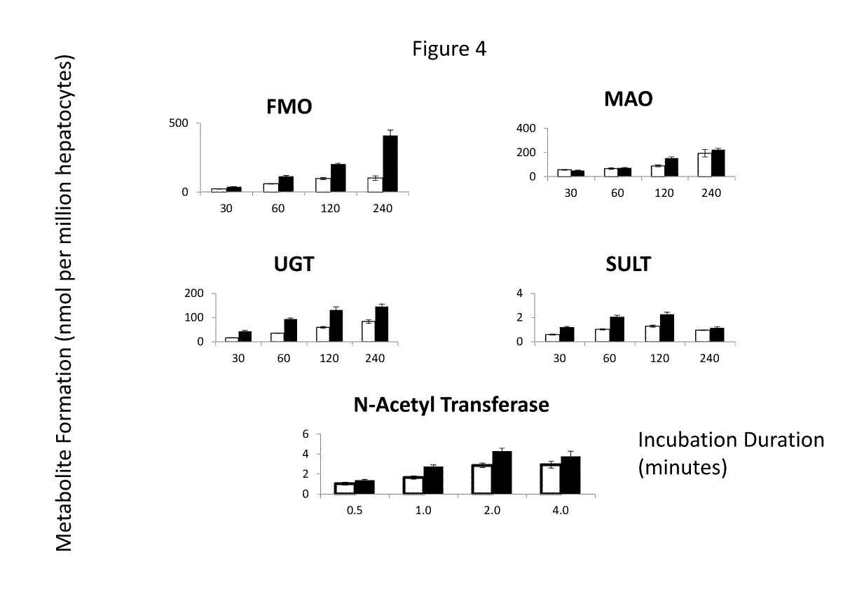

The results demonstrate a similar trend in the linear time course of metabolite formation in both the intact human hepatocytes and the present permeabilized hepatocytes. See FIG. 4

### Example 8: Evaluation of Coumarin Metabolism Demonstrating Sequential Metabolism: Phase 1 Oxidation Followed by Phase 2 Conjugation of Metabolites

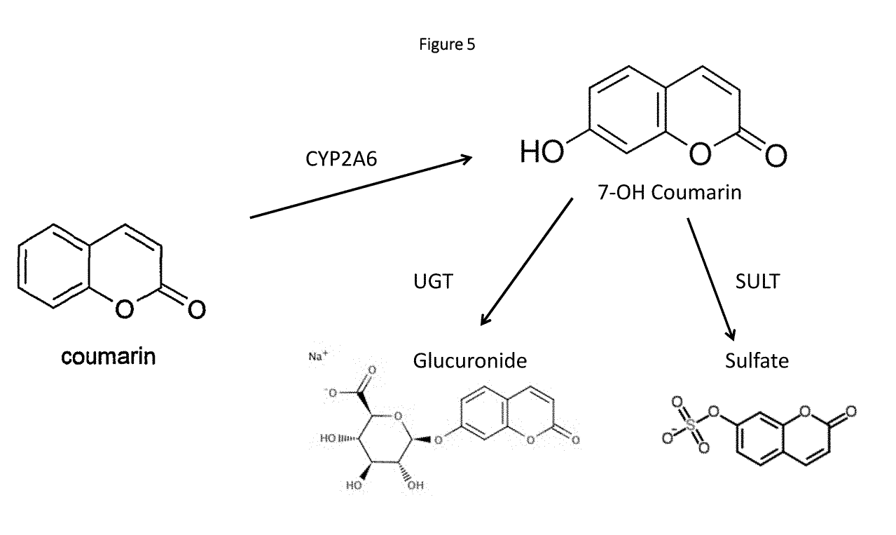

The assay was performed essentially as disclosed in Example 1. The cryopreserved permeabilized hepatocytes/cofactor mixture was thawed at 37° C. and 50 μL of the mixture was added to 50 μL of HQM containing 75 micromolar coumarin in wells of a 96-well plate. See FIG. 5 for a cartoon drawing of coumarin metabolism. The plate was incubated in a cell culture incubator maintained at 37° C. for 30 minutes after which an organic solvent (e.g. acetonitrile) as added at a volume of 100 μL, to each well. The contents of the wells were collected for LC/MS-MS quantification of metabolite formation (7-hydroxycoumarin and its conjugated metabolites, 7-glucuronide and 7-sulfate coumarin). The intact hepatocytes were thawed at 37° C., recovered by adding the thawed hepatocytes to UCRM, centrifuged at 100×g for 10 minutes, resuspended in HQM followed by microscopic evaluation of viability based on trypan blue, cell concentration adjusted with HQM to 2 million cells per mL, followed by incubation with drug metabolism substrates as described above for the permeabilized hepatocytes.

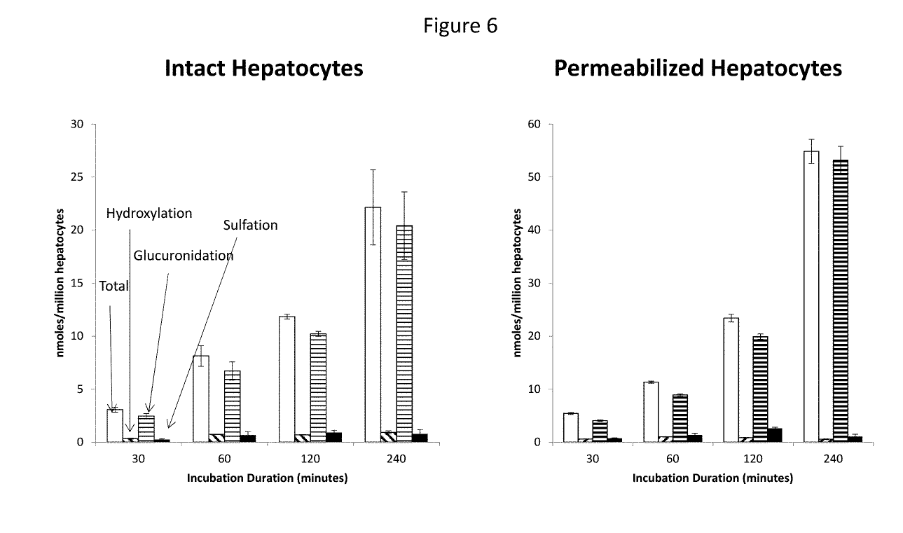

The results demonstrate the present permeabilized hepatocytes are capable of sequential drug metabolism, similar to intact human hepatocytes. See FIG. 6.

### Example 9: Evaluation of Drug Metabolizing Enzymes in Intact Human Hepatocytes and Present Permeabilized Hepatocytes

The permeabilized hepatocytes were prepared essentially as disclosed in Example 1, wherein the hepatocytes were pooled from multiple donors. The cryopreserved permeabilized hepatocytes/cofactor mixture was thawed at 37° C. and 50 μL of the mixture was added to 50 μL of HQM containing metabolic pathway-selective substrates (See Table 2 below). The plate was incubated in a cell culture incubator maintained at 37° C. for 30 minutes after which an organic solvent (e.g. acetonitrile) was added at a volume of 100 μL to each well. The contents of the wells were collected for LC/MS-MS quantification of metabolite formation. The intact hepatocytes were thawed at 37° C., recovered by adding the thawed hepatocytes to UCRM, centrifuged at 100×g for 10 minutes, resuspended in HQM followed by microscopic evaluation of viability based on trypan blue, cell concentration adjusted with HQM to 2 million cells per mL, followed by incubation with drug metabolism substrates as described above for the permeabilized hepatocytes.

TABLE 2
drug metabolizing enzyme substrates evaluated:

| Metabolic Pathway | Substrate | Substrate Conc. (μM) | Marker Metabolite |
|---|---|---|---|
| CYP1A2 | Phenacetin | 100 | Acetaminophen |
| CYP2A6 | Coumarin | 50 | 7-HC, 7-HC-Sulfate, 7- |
| | | | HC-Glucuronide |
| CYP2B6 | Buproprion | 500 | Hydroxybuproprion |
| CYP2C8 | Paclitaxel (Taxol) | 20 | 6α-hydroxypaclitaxel |
| CYP2C9 | Diclofenac | 25 | 4-OH Diclofenac |
| CYP2C19 | S-Mephenytoin | 250 | 4-OH S-Mephenytoin |
| CYP2D6 | Dextromethorphan | 15 | Dextrophan |
| CYP2E1 | Chlorzoxazone | 250 | 6-OH Chlorzoxazone |
| CYP3A4-1 | Midazolam | 20 | 1-Hydroxymidazolam |
| CYP3A4-2 | Testosterone | 200 | 6β-hydroxytestosterone |
| ECOD | 7-Ethoxycoumarin | 100 | 7-HC, 7-HC-Sulfate, 7- |
| | | | HC-Glucuronide |
| SULT | 7-Hydroxycoumarin | 100 | 7-Hydroxycoumarin |
| | | | Sulfate |
| UGT | 7-Hydroxycoumarin | 100 | 7-Hydroxycoumarin |
| | | | Glucuronide |
| GST | Acetaminophen | 10 mM | Acetaminophen |
| | | | Glutathione |
| FMO | Benzydamine HCl | 250 | Benzydamine-N-Oxide |
| MAO | Kynuramine HBr | 160 | 4-hydroxyquinoline |
| AO | Carbazeran | 10 | 4-Hydroxycarbazeran |

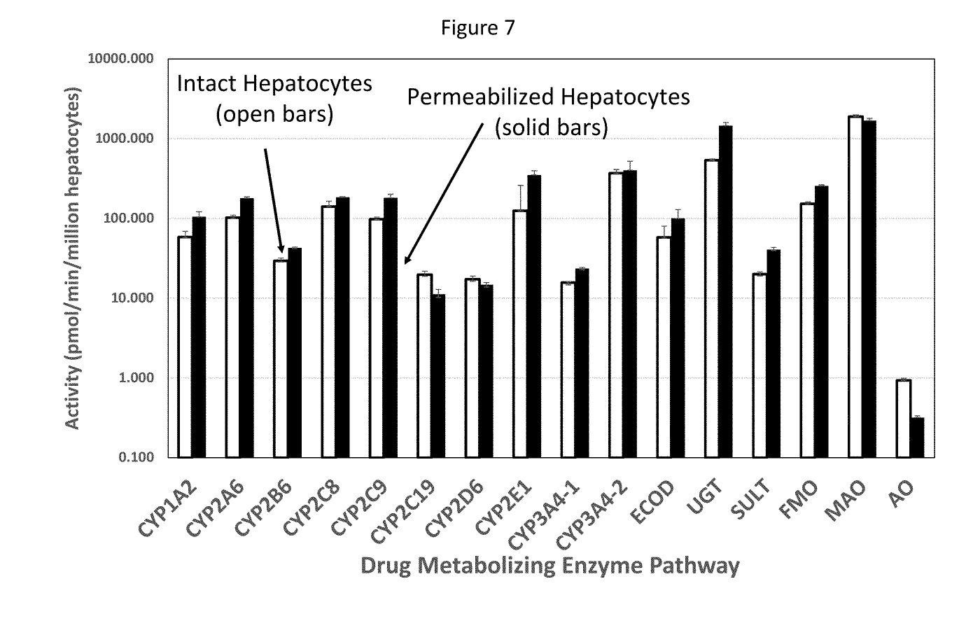

The results demonstrate the present permeabilized hepatocytes and intact human hepatocytes possess similar activities in the metabolism of 17 pathway-selective drug metabolizing enzyme pathways. See FIG. 7. The results suggest the present permeabilized human hepatocytes can be used in lieu of intact human hepatocytes for drug metabolism studies. The convenience of the permeabilized human hepatocytes allows it to be a more efficient experimental system than the conventional human intact hepatocytes.

### Example 10: Evaluation of Drug Metabolizing Enzymes in Intact Human Enterocytes and Present Permeabilized Enterocytes

The permeabilized enterocytes were prepared essentially as disclosed in Example 2, wherein the enterocytes were pooled from multiple donors. The cryopreserved permeabilized enterocytes/cofactor mixture was thawed at 37° C. and 50 μL of the mixture was added to 50 μL of HQM containing metabolic pathway-selective substrates (See Table 3). The plate was incubated in a cell culture incubator maintained at 37° C. for 30 minutes after which an organic solvent (e.g. acetonitrile) was added at a volume of 100 μL to each well. The contents of the wells were collected for LC/MS-MS quantification of metabolite formation. The intact enterocytes were thawed at 37° C., recovered by adding the thawed hepatocytes to Cryopreserved Enterocytes Recovery Medium, centrifuged at 100×g for 10 minutes, resuspended in HQM followed by microscopic evaluation of viability based on trypan blue, cell concentration adjusted with HQM to 2 million cells per mL, followed by incubation with drug metabolism substrates as described above for the permeabilized enterocytes.

The following drug metabolizing enzyme substrates (See Table 3) were evaluated in intact human enterocytes, pooled intact human enterocytes and permeabilized enterocytes and the metabolite activity measured. The results demonstrate the present permeabilized human enterocytes are equal or more active than the intact human enterocytes in all pathways evaluated.

TABLE 3
Enhanced drug metabolism enzyme activities of
permeabilized cryopreserved human enterocytes

| | | | Metabolic Activity (pmol/10⁶/min) | | |
|---|---|---|---|---|---|
| | | | | Intact | Permeabilized |
| Metabolic | | Marker | Pooled Intact | Cryopreserved | Cryopreserved |
| Pathway | Substrate | Metabolite | Enterocytes | Enterocytes | Enterocytes |
| CYP2C9 | Diclofenac | 4-OH Diclofenac | 4.05 ± 0.16 | 2.09 ± 0.26 | 5.78 ± 1.13 |
| CYP2C19 | S- | 4-OH S- | 0.55 ± 0.03 | 0.35 ± 0.11 | 3.36 ± 0.32 |
| | Mephenytoin | Mephenytoin | | | |
| CYP3A4- | Midazolam | 1-OH-midazolam | 1.21 ± 0.03 | 0.56 ± 0.05 | 4.23 ± 1.22 |
| 1 | | | | | |
| CYP3A4- | Testosterone | 6βOH- | 10.6 ± 3.3 | 10.1 ± 2.59 | 147 ± 14.5 |
| 2 | | testosterone | | | |
| UGT | 7-OH- | 7- | 16.05 ± 0.32 | 6.31 ± 0.43 | 275 ± 79.5 |
| | Coumarin | Hydroxycoumarin | | | |
| | | Glucuronide | | | |
| SULT | 7-OH- | 7- | 7.24 ± 0.34 | 4.64 ± 0.15 | 13 ± 0.69 |
| | Coumarin | Hydroxycoumarin | | | |
| | | Sulfate | | | |
| 2J2 | Astemizole | O-Demethyl | 0.92 ± 0.43 | BLQ | 5.14 ± 1.53 |
| | | Astemizole | | | |
| CES2 | Irinotecan | SN38 | 0.37 ± 0.14 | 0.03 ± 0.07 | 0.38 ± 0.27 |

### Example 11: Metabolic Stability Screening—Evaluation of Intrinsic Hepatic Clearance

Eighteen drugs (See Table 4) with known in vivo hepatic clearance were evaluated in both intact human hepatocytes and the present permeabilized hepatocytes, which were prepared essentially as disclosed in Example 1 wherein the hepatocytes from multiple donors were pooled. The drugs evaluated were added at a concentration of 1 μM to a hepatocyte culture of 1 million cells/ml. The cell culture was incubated and metabolites measured at time points: 0, 5, 15, 30 and 45 minutes and in vivo clearance measured. See Table 4, wherein in vivo clearance and predicted clearance using intact human hepatocytes and the present permeabilized hepatocytes are provided.

TABLE 4
Evaluation of drugs with known in vivo hepatic clearance

| | | Observed | | | | |
|---|---|---|---|---|---|---|
| | Enzymes | in vivo | Intact | | Permeabilized | |
| | Responsible | CI_{sys} | Hepatocytes | | Hepatocytes | |
| Drug | for Metabolism | (mL/min/kg) | Average | STDEV | Average | STDEV |
| Acebutolol | Acetylation | 5.2 | 2.2 | 0.5 | 1.2 | 0.3 |
| Afatinib | FMO/GST | 15 | 3.3 | 1.7 | 16.2 | 0.7 |
| Antipyrine | CYP1A2 | 0.5 | 1.3 | 0.9 | 1.8 | 0.3 |
| Betaxolol | P450 | 4.8 | 3.0 | 0.9 | 1.3 | 0.7 |
| Carbamazepine | CYP3A4 > 2C8/9 | 0.4 | 1.0 | 0.5 | 1.0 | 0.3 |
| Chlorzoxazone | CYP2E1 | 5.1 | 5.3 | 1.4 | 5.2 | 1.3 |
| Clozapine | CYP1A2 >> UGT, | 6.1 | 4.6 | 1.4 | 3.2 | 3.1 |
| | 2D6, 3A4 | | | | | |
| Desipramine | CYP2D6 > UGT | 10.3 | 3.1 | 0.1 | 5.0 | 1.4 |
| Dextromethorphan | CYP2D6 > 3A/2C19 | 8.6 | 10.3 | 0.6 | 7.3 | 0.7 |
| Diazepam | CYP2C19 > 3A | 0.4 | 3.7 | 0.5 | 2.5 | 0.9 |
| Diclofenac | CYP2C9 | 4.2 | 13.9 | 0.8 | 16.9 | 0.5 |
| Diltiazem | CYP3A4 | 12 | 8.2 | 0.5 | 4.8 | 1.8 |
| Furosemide | P450 | 1.7 | 4.8 | 2.4 | 2.0 | 1.7 |
| Imipramine | CYP2D6/1A2/2C19/ | 8 | 10.5 | 0.3 | 8.9 | 0.6 |
| | 3A/UGT1A4 | | | | | |
| Lorazepam | UGT | 1.1 | 2.0 | 1.0 | 0.7 | 0.6 |
| Nifedipine | CYP3A4 | 15 | 10.5 | 0.2 | 10.4 | 1.0 |
| Propranolol | CYP2D6 > | 13 | 10.7 | 0.4 | 7.6 | 0.7 |
| | 1A2/2C19/UGT | | | | | |
| Verapamil | CYP3A4 | 14 | 13.6 | 0.5 | 12.9 | 1.0 |
| Warfarin | CYP2C9, 3A4 | 0.05 | 2.9 | 0.9 | 1.5 | 0.8 |

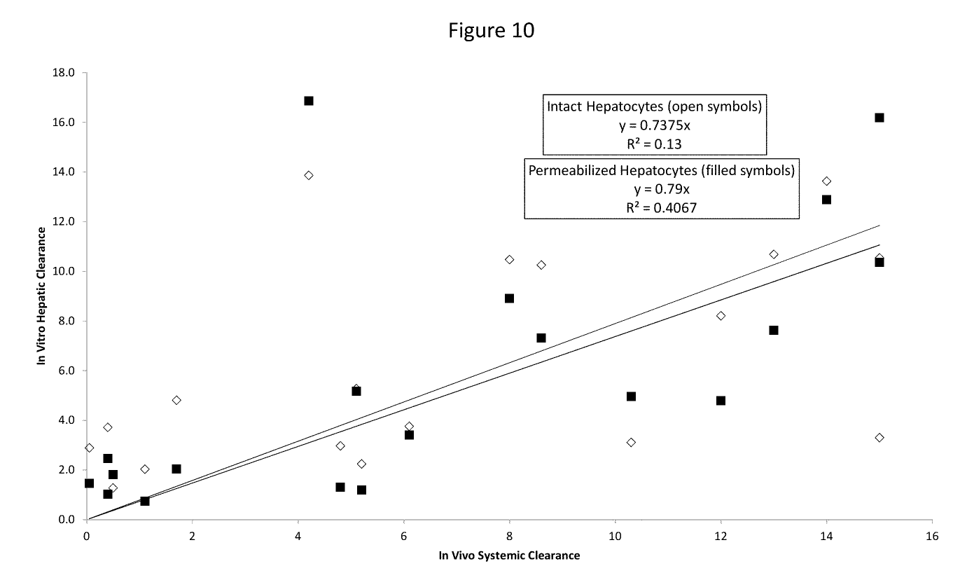

The results demonstrated a linear correlation with in vivo hepatic clearance, wherein a similar result is shown for both the intact human hepatocytes and the present permeabilized human hepatocytes. However, the present permeabilized human hepatocytes yield a higher coefficient of correlation than intact human hepatocytes. See FIG. 10.

### Example 12: Metabolite Profiling—Metabolite Formation at Cytotoxic Drug Concentrations

Human hepatocytes, with complete drug metabolizing enzyme (DME) pathways, should be ideal for metabolite profiling studies. However, due to cytotoxicity, hepatocytes in general cannot be incubated with high drug concentrations to allow the production of adequate quantity of metabolites for identification.

The permeabilized hepatocytes were prepared essentially as disclosed in Example 1, wherein the hepatocytes were pooled from multiple donors. The cryopreserved permeabilized hepatocytes/cofactor mixture was thawed at 37° C. and 50 μL of the mixture was added to 50 μL of HQM containing a noncytotoxic concentration (10 mM), and two cytotoxic concentrations (100 and 200 mM) of acetaminophen (APAP) in wells of a 96-well plate. The plate was incubated in a cell culture incubator maintained at 37° C. for 30 minutes after which an organic solvent (e.g. acetonitrile) as added at a volume of 100 μL to each well. The contents of the wells are collected for LC/MS-MS quantification of metabolite formation (glucuronide, sulfate, and glutathione conjugated acetaminophen). The intact hepatocytes were thawed at 37° C., recovered by adding the thawed hepatocytes to UCRM, centrifuged at 100×g for 10 minutes, resuspended in HQM followed by microscopic evaluation of viability based on trypan blue, cell concentration adjusted with HQM to 2 million cells per mL, followed by incubation with drug metabolism substrates as described above for the permeabilized hepatocytes.

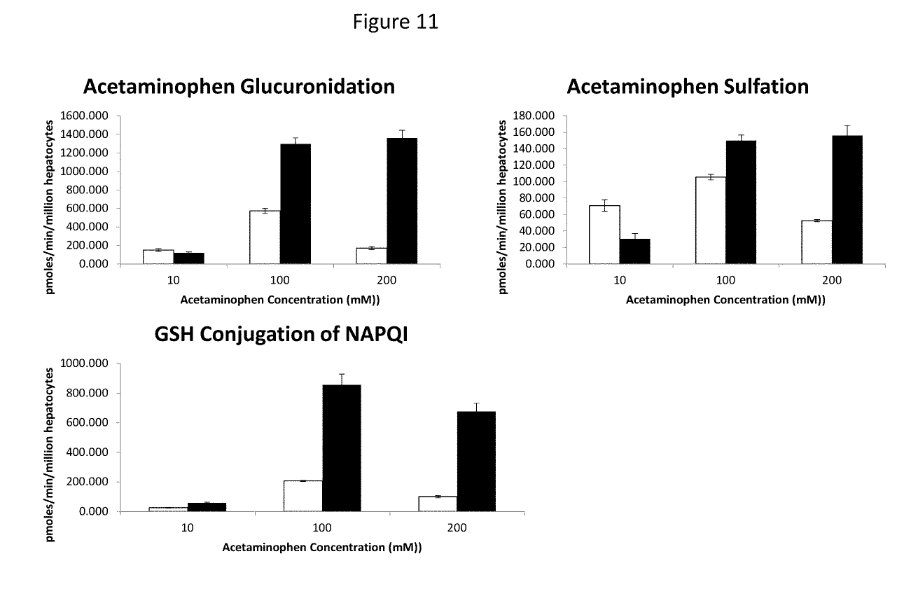

The results demonstrate a similar metabolite profiles for glucuronide, sulfate and GDH conjugate at the noncytotoxic (10 mM) concentration. However, the present permeabilized human hepatocytes demonstrated an enhanced metabolite formation as compared to intact human hepatocytes at cytotoxic concentrations (100 and 200 mM) of acetaminophen. See FIG. 11. These results suggest the present permeabilized human hepatocytes can be incubated with high, cytotoxic concentrations of a xenobiotic, such as a drug, for the generation of sufficient amounts of metabolites for identification and profiling.

### Example 13: Cofactor-Directed Metabolic Pathway Selection

Intact human hepatocytes allow metabolism of a drug by all hepatic pathways, selection of a specific metabolic pathway for evaluation is not easily accomplished. However, the experiment disclosed herein demonstrates the present permeabilized human hepatocytes can be used to direct metabolism to specific pathways via selection of cofactors (e.g., exogenous drug metabolizing enzyme co-factors).

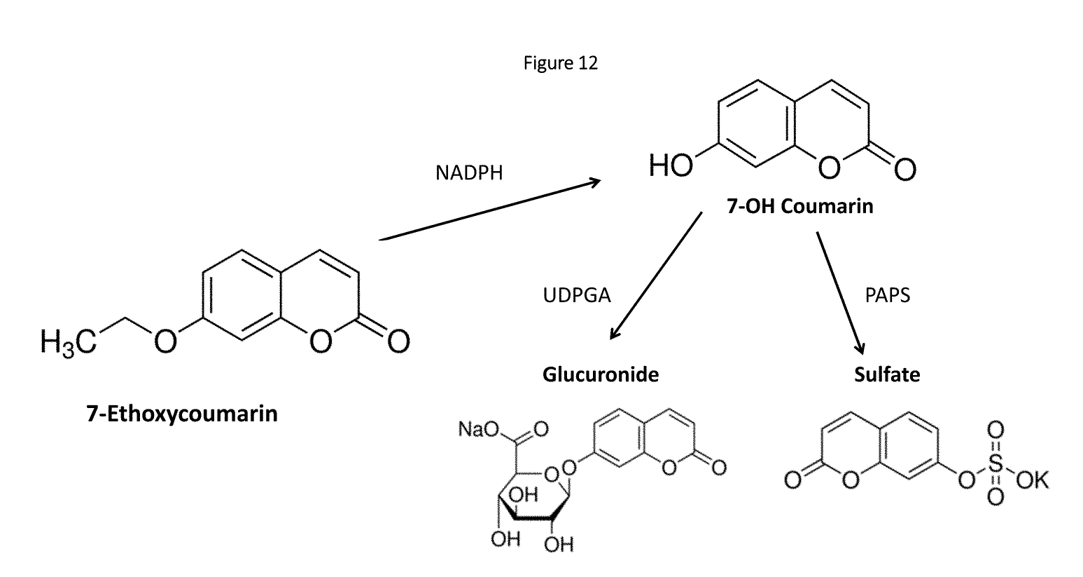

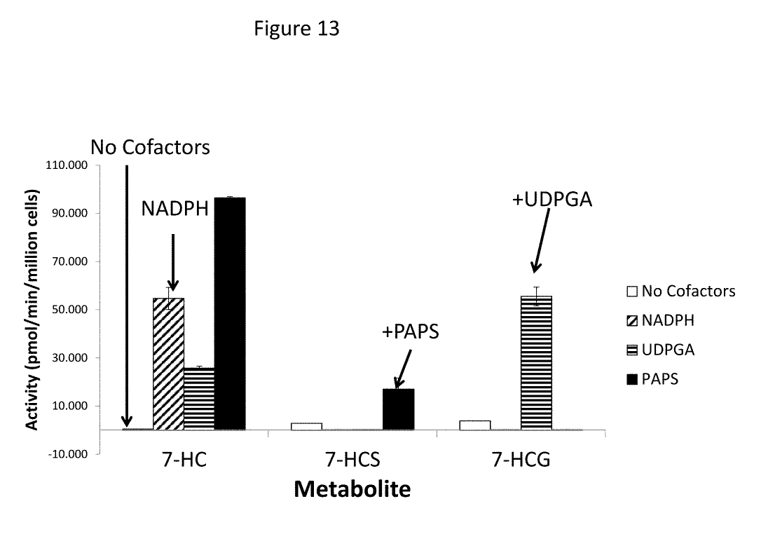

The metabolism of coumarin was used as a model for cofactor directed metabolic pathway selection. See FIG. 12 for the co-factor directed pathway selection of coumarin metabolism with the present permeabilized human hepatocytes. The exogenous drug metabolizing enzyme co-factors used were 2 mM NADPH, 2 mM NADPH+1 mM PAPS and 2 mM NADPH+5 mM UDGPGA, and no cofactor addition used as a control. The cryopreserved permeabilized hepatocytes with the various cofactor compositions were thawed at 37° C. and 50 μL of the mixture was added to 50 μL of HQM containing 75 micromolar of coumarin. The plate is incubated in a cell culture incubator maintained at 37° C. for 30 minutes after which an organic solvent (e.g. acetonitrile) as added at a volume of 100 μL to each well. The contents of the wells were collected for LC/MS-M S quantification of metabolite formation. The metabolites 7-OH coumarin (7-HC), 7-HC-sulfate (7-HCS) and 7-HC-glucuronide (7-HCG) were measured. See FIG. 13.

TABLE 5
Cofactor selection

| Cofactors | Expectation | Result |
|---|---|---|
| No co-factors | No metabolism | No metabolism |
| NADPH only | 7-OH coumarin (7-HC) formation; | 7-OH coumarin (7-HC) formation; |
| | no sulfation of glucuronidation | minimal sulfation and glucuronidation |
| NADPH + | 7-HC and 7-HC-glucuronide | 7-HC and 7-HC-glucuronide |
| UDPGA | formation; no 7-HC-sulfate | formation; no 7-HC-sulfate |
| NADPH + | 7-HC and 7-HC-sulfate formation; | 7-HC and 7-HC-sulfate formation; no |
| PAPS | no glucuronide | glucuronide |

### Example 14: Evaluation of the Present Permeabilized Human Hepatocytes as Exogenous Metabolic Activation System for the Evaluation of Prototoxicants

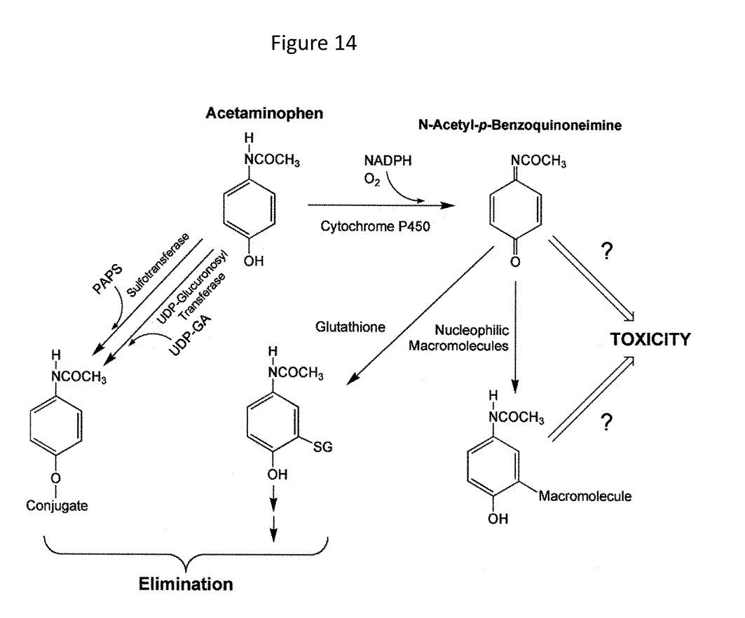

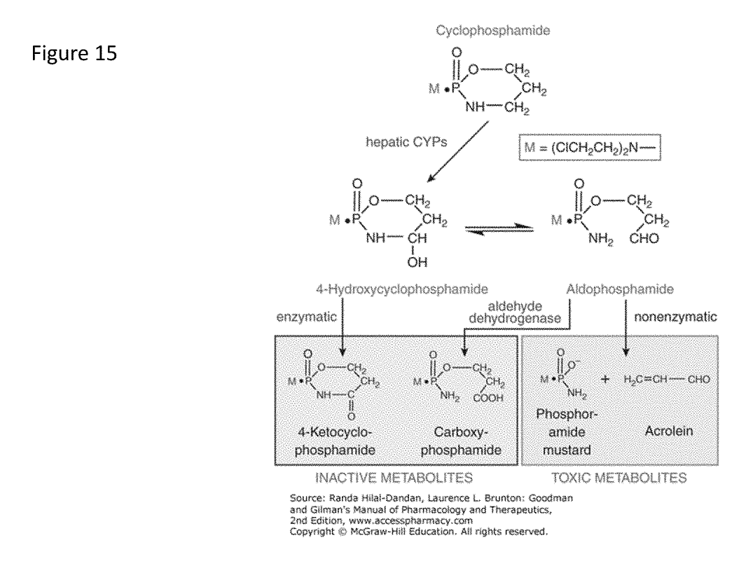

Acetaminophen (See FIG. 14), Cyclophosphamide (See FIG. 15) and Ifosfamide were evaluated in the following prototoxicant activation assay. In this assay permeabilized human hepatocytes were used in an exogenous metabolic system for evaluation of the physiological effect of metabolites of the prototoxicants towards a target cell line, the HEK293 cells which are devoid of xenobiotic drug metabolism activities. Inactivated (boiled) permeabilized human hepatocytes were used as a metabolic negative control.

In this assay, HEK293 cells were cultured in DMEM in a 96-well plate (10,000 cells per well; 50 μL) followed by addition of an equal volume of various concentrations of the prototoxicants acetaminophen, cyclophosphamide, and ifosfamide and an equal volume of medium containing permeabilized human hepatocytes (cofactor supplemented), boiled permeabilized hepatocytes or no hepatocytes. The cell cultures were incubated in a cell culture incubator maintained in a 37 C highly humidified atmosphere of 5% carbon dioxide and 95% air for 24 hours. After incubation, cellular ATP content of the HEK293 cells in each well were quantified using Promega Glo™, a luminescent ATP quantification reagent (Promega Inc., Madison, Wis.), with luminescence quantified using a Perkin Elmer Victor 3 plate reader.

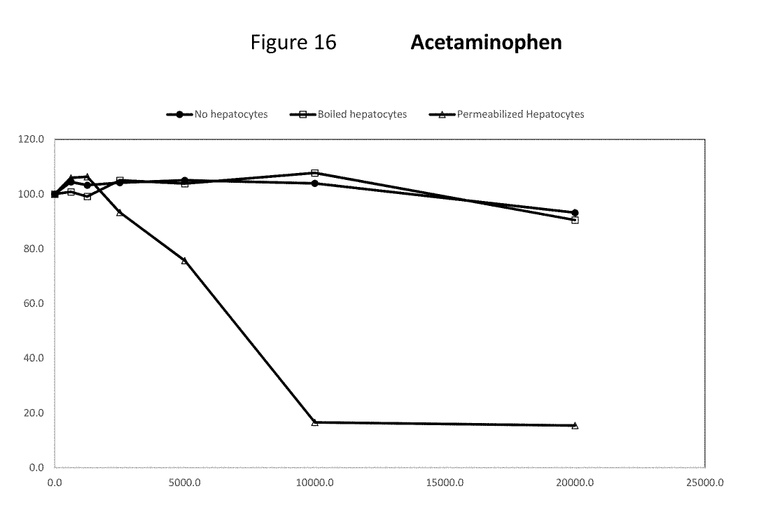

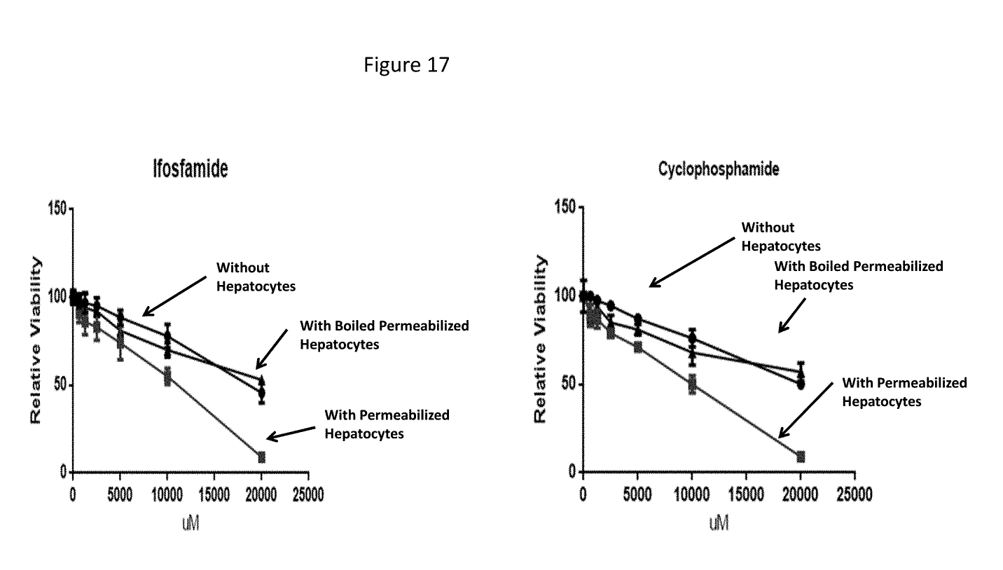

The results demonstrate the cytotoxicity of Acetaminophen, Cyclophosphamide and Ifosfamide on HEK 293 cells was enhanced by the present permeabilized human hepatocytes, wherein the present permeabilized human hepatocytes can be used as an exogenous activating system for the evaluation of prototoxicants. See FIG. 16 for activation of Acetaminophen and FIG. 17 for activation of Ifosfamide and Cyclophosphamide.

### Example 15: Evaluation of the Inhibition of Intestinal CYP3A4 from Orally Administered Drugs—Drug-Drug Interactions and Food-Drug Interactions

Permeabilized cofactor supplemented enterocytes were prepared from cryopreserved intact enterocytes from 10 donors, see Example 2. Effects of herbal supplements on enterocyte CYP3A4 activity was evaluated using luciferin IPA as CYP3A4 substrate (LIPA; Promega, Madison, Wis.) with luminescence quantified on a Perkin Elmer Wallac 1420 Victor microplate reader. The herbal supplements were obtained commercially. The daily recommended dose was dissolved in 50 mL (4× of 100% concentration) of HQM (IVAL), pH adjusted to 7.0 to 7.2, and sterilized by filtration. For the drug interaction studies, aliquots of 50 μL of permeabilized enterocyte suspension per well were added to 96-well plates, followed by an addition of 25 μL of the 4× herbal supplements and 25 μL of 4×LIPA. CYP3A4 activity was quantified after an incubation period of 60 minutes.

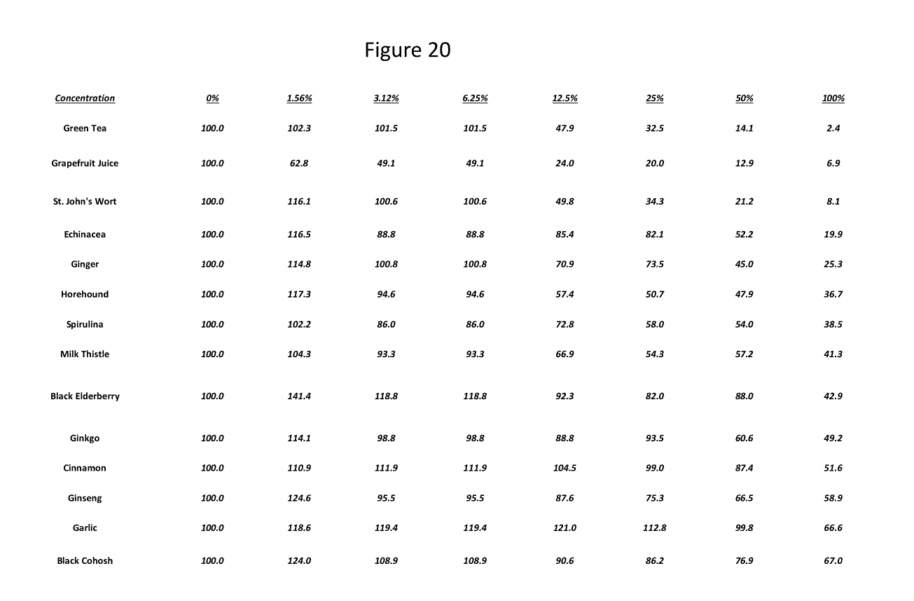

The results are shown in FIG. 20. The results show that permeabilized enterocytes can be used for the evaluation of food-drug interactions. Herbal supplements with significant CYP3A4 inhibitory effects (e.g. green tea extract; grapefruit juice) are likely to inhibit enteric metabolism of orally-administered drugs that are CYP3A4 substrates.

Those skilled in the art can devise many modifications and other embodiments within the scope and spirit of the presently disclosed inventions. Indeed, variations in the materials, methods, drawings, experiments examples and embodiments described may be made by skilled artisans without changing the fundamental aspects of the disclosed inventions. Any of the disclosed embodiments can be used in combination with any other disclosed embodiment.

The disclosed embodiments, examples and experiments are not intended to limit the scope of the disclosure nor to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

1. An in vitro reagent for evaluating xenobiotic metabolism, wherein the reagent is a cryopreserved mixture comprising:
a) metabolically competent cells wherein cell membranes are permeabilized upon thawing;
b) exogenous drug metabolizing enzyme co-factors, and
c) a buffered solution,
wherein the reagent does not comprise a cryopreservative agent.

2. The reagent of claim 1, wherein the metabolically competent cells are hepatocytes or enterocytes.

3. (canceled)

4. The reagent of claim 1, wherein the metabolically competent cells are engineered to contain cytochrome P450 isoforms.

5. The reagent of claim 1, wherein the metabolically competent cells comprise drug metabolizing enzyme (DME) activities.

6-9. (canceled)

10. The reagent of claim 1, wherein the drug metabolizing enzyme co-factors are selected from β-Nicotinamide adenine dinucleotide 2′-phosphate (NADPH), Uridine 5′-diphosphoglucuronic acid (UDPGA), 3′-Phosphoadenosine 5′-phosphosulfate (PAPS), N-acetyl coenzyme A, s-adenosyl methionine, amino acids, carnitine, and L-glutathione.

11. The reagent of claim 1, wherein the reagent is stored frozen at a temperature of −10° C. to about −80° C.

12. The reagent of claim 11, wherein the reagent is thawed prior to use for evaluating xenobiotic metabolism.

13. The reagent of claim 1, wherein the reagent is further added to and comprises a mammalian or non-mammalian culture, wherein the reagent provides exogenous metabolic functions for evaluation of a physiological effect of metabolites of a xenobiotic.

14. The reagent of claim 13, wherein the non-mammalian culture comprises Salmonella bacteria used in an Ames Salmonella histidine reversion assay (Ames test) for genotoxicity.

15. The reagent of claim 13, wherein the mammalian or non-mammalian culture comprises cells used in a genotoxicity assay.

16. The reagent of claim 13, wherein the mammalian or non-mammalian culture comprises cells used in a cytotoxicity assay.

17. The reagent of claim 13, wherein the mammalian or non-mammalian culture comprises cells used in a pharmacological assay.

18. The reagent of claim 1, wherein the reagent is provided in a kit and further comprises instructions for evaluating xenobiotic metabolism using the reagent.

19. (canceled)

20. (canceled)

21. A method for manufacturing an in vitro reagent for evaluating xenobiotic metabolism, comprising:
a) permeabilizing metabolically competent cells;
b) adding exogenous drug metabolizing enzyme co-factors either before or after the metabolically competent cells are permeabilized; and
c) providing a buffered solution,
wherein the reagent does not comprise a cryopreservative agent.

22. The method of claim 21, wherein the reagent is further stored frozen at a temperature of −10° C. to about −80° C.

23. (canceled)

24. The method of claim 21, wherein the metabolically competent cells are permeabilized by freezing and thawing in the absence of a cryopreservative agent.

25-46. (canceled)

47. A method for evaluating xenobiotic metabolism, comprising:
a) providing the cryopreserved in vitro reagent of claim 1;
b) thawing the in vitro reagent and placing in a cell culture vessel;
c) introducing the xenobiotic test compound into the cell culture vessel;
d) incubating the test compound for 0.5 h to 10 days at 33-40° C.; and,
e) performing an end point assay of the in vitro reagent or cell culture medium to determine metabolism of the xenobiotic test compound.

48-52. (canceled)

53. The method of claim 47, wherein the xenobiotic agent is a drug or drug candidate, an industrial chemical, or an environmental pollutant.

54. (canceled)

55. (canceled)

56. The method of claim 53, wherein the drug or drug candidate is selected from the group consisting of an organic compound, an inorganic compound, a hormone, a growth factor, a cytokine, a reception, an antibody, an enzyme, a peptide, an aptomer and a vaccine.

57. The method of claim 47, wherein step b) does not further comprise a centrifugation or cell counting step.

58-78. (canceled)
